(19) United States
(12) Patent Application Publication
(10) Pub. No.: US 2024/0425890 A1
(43) Pub. Date: Dec. 26, 2024

(54) PROCESS FOR THE SYNTHESIS OF ALPHA-METHYLENE-GAMMA-BUTYROLACTONE

(71) Applicant: Henkel AG & Co. KGaA, Duesseldorf (DE)

(72) Inventors: Andreas Taden, Duesseldorf (DE); Horst Beck, Neuss (DE); Kamela Myrtollari, Duesseldorf (DE); Robert Kourist, Graz (AT); Andrea Nigl, Graz (AT)

(21) Appl. No.: 18/800,537

(22) Filed: Aug. 12, 2024

Related U.S. Application Data
(63) Continuation of application No. PCT/EP2023/050793, filed on Jan. 16, 2023.

(30) Foreign Application Priority Data
Feb. 11, 2022 (EP) 22156336.4

(51) Int. Cl. C12P 17/04; C12N 9/02; C12N 9/04

(52) U.S. Cl. CPC C12P 17/04; C12N 9/0006; C12N 9/0008; C12N 9/0061; C12N 9/0077; C12Y 101/01001; C12Y 101/03013; C12Y 102/01003; C12Y 114/15003

(57) ABSTRACT

The present application provides a process for the preparation of α-methylene-γ-butyrolactone, said process comprising the steps of:
a) acetylating the C1-hydroxyl group of isoprenol to yield isoprenyl acetate;
b) forming 4-acetoxy-2-methylene-butan-1-ol from said isoprenyl acetate by whole cell biotransformation, said step comprising:
   i) contacting a cell (CB) with a culture medium containing said isoprenyl acetate or with a culture medium contiguous with an organic phase containing said isoprenyl acetate under conditions that enable the cell to form 4-acetoxy-2-methylene-butan-1-ol from isoprenyl acetate; and,
   ii) optionally isolating the resultant 4-acetoxy-2-methylene-butan-1-ol, wherein said cell (CB) exhibits activity of at least one alkane monooxygenase enzyme which catalyzes the formation of 4-acetoxy-2-methylene-butan-1-ol from isoprenyl acetate;
c) oxidizing said 4-acetoxy-2-methylene-butan-1-ol to yield 4-acetoxy-2-methylene butyric acid; and,
d) converting said 4-acetoxy-2-methylene butyric acid to α-methylene-γ-butyrolactone by hydroxylysis to γ-hydroxy-α-methylenebutyric acid and subsequent cyclization of said γ-hydroxy-α-methylenebutyric acid.

### FIELD OF THE INVENTION

The present disclosure is directed to a process for the synthesis of α-methylene-γ-butyrolactone (MBL) from isoprenol. More particularly, the present disclosure is directed to a multi-step process for the synthesis of α-methylene-γ-butyrolactone (MBL) from isoprenol, of which at least one step comprises whole cell biotransformation and in which process a number of important intermediate compounds are identified which have utility in their own right.

### BACKGROUND TO THE INVENTION

Significant research is being undertaken to develop renewable or sustainable polymeric materials which can replace petroleum based raw materials for large commodity and specialty chemical markets. Bio-derived polylactides and polyhydroxyalkanoates (PHA)—such as poly-3-hydroxybutyrate (P3HB), poly-4-hydroxybutyrate (P4HB), polyhydroxyvalerate (PHV), polyhydroxyhexanoate (PHH), polyhydroxyoctanoate (PHO) and the copolymers thereof—have long garnered attention in this regard. In contrast, unsaturated lactones, which can be isolated from plant sources or derived from itaconic or levulinic acids obtained in the microbial fermentation of biomass feedstocks, have been less extensively studied to date.

An advantage of unsaturated lactones is that they carry two different functional moieties—specifically vinyl and lactone—in one molecule, both said moieties being polymerizable. Thus, these monomers can have utility in the replacement of (meth)acrylates in vinyl-addition polymerization or as (co)monomers for preparation of degradable polyesters via lactone ring-opening polymerization (ROP). Using these unsaturated monomers—of which particular mention may be made of α-methylene-γ-butyrolactone (MBL), β-hydroxy-α-methylene-γ-butyrolactone (H-MBL), β- and γ-methyl-α-methylene-γ-butyrolactone (β-MMBL, γ-MMBL) and angelica lactones (α- and β-AL)—functional polymers can be derived which bear either pendant double bonds, pendant lactone rings, or other pendant substituents allowing for various post-functionalizations.

The present disclosure is directed to a novel synthetic route for the production of α-methylene-γ-butyrolactone (MBL). In addition to being a platform monomer for the aforementioned homo- and copolymers, it is recognized that the MBL structural unit—in α-methylene-γ-butyrolactone itself and as a part of various sesquiterpenoids—exhibits multiple biological properties, including antibacterial, cytotoxic, anti-inflammatory, antioxidant, allergenic and antimicrobial activity.

U.S. Pat. No. 5,166,357 (Orlek et al.) discloses a synthetic route to α-methylene-γ-butyrolactone comprising a two-step sequence consisting of: i) the reaction of γ-butyrolactone with ethyl formate in the presence of base; and, ii) the refluxing of the resulting α-formyl-γ-butyrolactone sodium salt under nitrogen with paraformaldehyde in tetrahydrofuran. Distillation affords the desired α-methylene-γ-butyrolactone as a colorless oil.

U.S. Pat. No. 6,362,346 B1 (Coulson et al.) describes a process for preparing α-methylene-γ-butyrolactone comprising heating a mixture of a furoic acid selected from the group consisting of tetrahydro-3-furoic acid and esters of tetrahydro-3-furoic acid and a strong acid catalyst under conditions whereby α-methylene-γ-butyrolactone is formed.

WO2012/116977 (DSM IP Assets BV) describes a process for the preparation of 3-methylene-Y-butyrolactone (Z), the process comprising: i) a hydroformylation step wherein 1,4-butene-diol (X) or its ester derivative cis-1,4-diacetoxy butene (Y) is subjected to H₂ gas and CO gas in the presence of a hydroformylation catalyst, thereby forming an intermediate product comprising a mixture of compounds containing an aldehyde group or a hemiacetal; and, ii) an oxidation step wherein the intermediate product or hydrolyzed derivative thereof is oxidized by an oxidation agent thereby forming Z.

In the described embodiments of WO2012/116977, the intermediate product is a mixture of hydroxy-2-(hydroxymethyl)butanal (X1), 4-hydroxy-2-methylenebutanal (X2), 3-(hydroxymethyl) tetrahydrofuran-2-ol (X3) and 3-methylenetetrahydrofuran-2-ol (X4). Whilst yields of these intermediates are not disclosed, it is submitted that 1,4-butene-diol (X) cannot be efficiently transformed thereto due to intermolecular acetalization, and this low efficiency will translate to a low yield of Z. Further, the formation of Z from said intermediates would not be efficient: even under base catalysis, there are competing reactions, in particular: the cyclization and dehydration reactions of X1 and X3; the reduction of X1 and X2 to the corresponding alcohol by H₂; and, the hydrogenation of X1, X2, X4 and Z to form the corresponding alkane. Such side reactions might be minimized through the use of the protected starting material cis-1,4-diacetoxy butene (Y) but this has disadvantages, such as the additional cost of the starting material, the longer reaction time for hydroformylation and the unfavorable molecular economy due to wasting protective groups.

The skilled artisan would further note that the process of WO2012/116977 relies on the use of explosive syngas under harsh conditions of temperature and pressure in the presence of transition metal catalyst having toxic phosphorus ligands. It is known that syngas is produced by the high temperature treatment of fossil-based carbon resources. Moreover, natural resources for transition metal catalyst based on Co, Rh, and Pd are limited geopolitically.

Trotta et al., Synthesis of methylene butyrolactone polymers from itaconic acid, J. Polym. Sci., Part A: Polym. Chem. 2017, 55, 2730-2737 reports the transformation of β-monomethyl itaconate, obtainable as a biorenewable feedstock, to α-methylene-γ,γ-dimethyl-γ-butyrolactone (Me₂MBL) and α-methylene-γ-butyrolactone (MBL, tulipalin A) through a selective addition strategy.

WO 2002101013 A2 (DuPont) describes the cloning of genes that encode proteins that are stated to be involved in the natural biosynthesis of α-methylene-γ-butyrolactone (Tulipalin A) and its intermediates. The citation more specifically discloses an isolated nucleic acid fragment encoding a tuliposide A synthesizing protein selected from the group consisting of: (a) an isolated nucleic acid fragment encoding one amino acid sequence set forth in SEQ ID NO:2, SEQ ID NO:4, SEQ ID NO:6, SEQ ID NO:8, SEQ ID NO:10, SEQ ID NO:14, SEQ ID NO:18, SEQ ID NO:20, SEQ ID NO:22 and SEQ ID NO:24 of this citation; (b) an isolated nucleic acid fragment that hybridizes with the aforementioned sequences under the following hybridization conditions: 0.1×SSC, 0.1% SDS at 65° C., and washed with 2×SSC, 0.1% SDS followed by 0.1×SSC, 0.1% SDS; and, (c) an isolated nucleic acid fragment that is completely complementary to (a) or (b). The proposed biosynthetic pathway of this citation does not include a hydratase step or any other enzymatic reactions leading to the intermediate product γ-methylene glutamate: for this reason alone, it is considered that the pathway is either not feasible or is otherwise incompletely or insufficiently disclosed in this citation.

WO2001068803A2 (Maxygen Inc.) describes methods for producing enzymes, biochemical pathways, and whole cell bioprocesses that convert simple carbon sources into polymerizable substrates, and into polyhydroxyalkanoates (PHAs). In particular, an artificial pathway for the synthesis of α-methylene-γ-hydroxybutyric acid in the itaconate producing fungus Aspergillus terreus is described. The citation suggests that itaconate is converted to itaconyl-CoA by endogenous enzymes of the fungus, which itaconyl-CoA is then converted to α-methylene succinate semialdehyde and further to α-methylene-γ-hydroxybutyric acid. These reductive steps are suggested to be catalyzed by a succinate-semialdehyde dehydrogenase and a γ-hydroxybutyrate dehydrogenase from Clostridium kluyveri, respectively. Whilst further potential enzymes which might be capable of catalyzing these reactions are listed in Table 1 of this citation, no related experimental data is provided. As many enzymes from strictly anaerobic organisms are highly sensitive to oxygen, an implementation into an aerobic organism is considered to be problematic.

The present inventors consider that a need exists in the art to provide a novel synthetic pathway to α-methylene-γ-butyrolactone (Tulipalin A) starting from a feedstock which may be biorenewable.

### STATEMENT OF THE INVENTION

In accordance with a first aspect of the present disclosure there is provided a process for the preparation of α-methylene-γ-butyrolactone, said process comprising the steps of:
a) acetylating the C1-hydroxyl group of isoprenol to yield isoprenyl acetate;
b) forming 4-acetoxy-2-methylene-butan-1-ol from said isoprenyl acetate by whole cell biotransformation, said step comprising:
   i) contacting a cell (CB) with a culture medium containing said isoprenyl acetate or with a culture medium contiguous with an organic phase containing said isoprenyl acetate under conditions that enable the cell to form 4-acetoxy-2-methylene-butan-1-ol from isoprenyl acetate; and,
   ii) optionally isolating the resultant 4-acetoxy-2-methylene-butan-1-ol, wherein said cell (CB) exhibits activity of at least one alkane monooxygenase enzyme which catalyzes the formation of 4-acetoxy-2-methylene-butan-1-ol from isoprenyl acetate;
c) oxidizing said 4-acetoxy-2-methylene-butan-1-ol to yield 4-acetoxy-2-methylene butyric acid; and,
d) converting said 4-acetoxy-2-methylene butyric acid to α-methylene-γ-butyrolactone by hydrolysis to γ-hydroxy-α-methylenebutyric acid and subsequent cyclization of said γ-hydroxy-α-methylenebutyric acid.

The isoprenol which is the starting point of this process step may be sourced commercially and, as such, could be obtained from the petroleum-derived chemicals isobutene and formaldehyde. However, it is preferred that the compound be sourced from a synthetic microbial system for the production of isoprenol.

The utilized cell (CB) of step b) possesses the gene of an alkane monoxygenase, optionally as part of an Alk operon. In an important embodiment, said cell (CB) of step b) is genetically modified to exhibit increased activity of an alkane monooxygenase enzyme encoded by a homologue of the AlkB gene from Pseudomonas putida GP01. Good results have been obtained wherein said cell (CB) is genetically modified to exhibit increased activity of an alkane monooxygenase enzyme encoded by an AlkB gene from Pseudomonas putida GP01 selected from the group consisting of: the AlkBGT gene cluster; the AlkBGTJH gene cluster; and, the AlkBGTJHL gene cluster.

Whilst step c) of the present disclosure may be performed chemically, in an important embodiment step c) is characterized by an enzymatic oxidation process comprising contacting 4-acetoxy-2-methylene butan-1-ol under aerobic conditions with: at least one enzyme exhibiting oxidizing activity; and, optionally, at least one mediating compound which enhances the oxidizing activity of the enzyme.

Step c) may equally be performed by whole cell biotransformation of which two important embodiments may be recognized.

In a first embodiment, step c) is performed by a whole cell biotransformation which comprises:
i) contacting a cell (CC) with a culture medium containing said 4-acetoxy-2-methylene-butan-1-ol or with a culture medium contiguous with an organic phase containing 4-acetoxy-2-methylene-butan-1-ol under conditions that enable the cell (CC) to form 4-acetoxy-2-methylene-butyric acid from 4-acetoxy-2-methylene-butan-1-ol; and,
ii) isolation of the resultant 4-acetoxy-2-methylene-butyric acid,
   wherein said cell (CC) exhibits activity of at least one enzyme exhibiting oxidizing activity. Preferably said at least one enzyme exhibiting oxidizing activity is selected from the group consisting of: alcohol dehydrogenase (ADH); alcohol oxidase (AlcOx); aldehyde dehydrogenases (AlDH); and, laccase.

In a second embodiment, step c) is performed by a whole cell biotransformation which comprises:
a) contacting a first cell (CC1) with a culture medium containing said 4-acetoxy-2-methylene-butan-1-ol or with a culture medium contiguous with an organic phase containing 4-acetoxy-2-methylene-butan-1-ol under conditions that enable the cell to form 4-acetoxy-2-methylene-butan-1-al from 4-acetoxy-2-methylene-butan-1-ol;
ii) optionally isolating the resultant 4-acetoxy-2-methylene-butan-1-al;
iii) contacting a second cell (CC2) with a culture medium containing said 4-acetoxy-2-methylene-butan-1-al or with a culture medium contiguous with an organic phase containing said 4-acetoxy-2-methylene-butan-1-al under conditions that enable the cell to form 4-acetoxy-2-methylene butyric acid from 4-acetoxy-2-methylene-butan-1-al; and,
iv) isolating the resultant 4-acetoxy-2-methylene butyric acid,

wherein said first and second cells (CC1, CC2) each exhibit activity of at least one enzyme exhibiting oxidizing activity. Preferably: said first cell (CC1) is genetically modified to exhibit increased activity of at least one of enzyme exhibiting oxidizing activity selected from the group consisting of alcohol dehydrogenase (ADH), alcohol oxidase (AlcOx) and laccase; and, said second cell (CC2) is genetically modified to exhibit increased activity of at least one of enzyme exhibiting oxidizing activity selected from the group consisting of aldehyde dehydrogenase (AlDH) and laccase.

As regards the aforementioned enzymes exhibiting oxidizing activity, it is particularly preferred that said alcohol dehydrogenase (ADH) enzyme is encoded by a homologue of the AlkJ gene from Pseudomonas putida GP01; and/or, said aldehyde dehydrogenase (AlDH) enzyme is encoded by a homologue of the AlkH gene from Pseudomonas putida G P01.

The present disclosure also provides for the use of the α-methylene-γ-butyrolactone (MBL) obtained in the above-described process as a monomer in either an anionic polymerization process to yield homo- or co-polymers (p-MBL) having pendant lactone rings in the polymer chain or in a ring opening polymerization process to yield a polyester.

In accordance with a second aspect of the disclosure there is provided a process for the preparation of 4-acetoxy-2-methylene-butan-1-ol, said process comprising the steps of:
a. acetylating the C1-hydroxyl group of isoprenol to yield isoprenyl acetate;
b. forming 4-acetoxy-2-methylene-butan-1-ol from said isoprenyl acetate by whole cell biotransformation, said step comprising:
   i) contacting a cell (CB) with a culture medium containing said isoprenyl acetate or with a culture medium contiguous with an organic phase containing said isoprenyl acetate under conditions that enable the cell to form 4-acetoxy-2-methylene-butan-1-ol from isoprenyl acetate; and,
   ii) isolating the resultant 4-acetoxy-2-methylene-butan-1-ol,

wherein said cell (CB) exhibits activity of at least one alkane monooxygenase enzyme which catalyzes the formation of 4-acetoxy-2-methylene-butan-1-ol from isoprenyl acetate.

The utilized cell (CB) of step b) possesses the gene of an alkane monoxygenase, optionally as part of an Alk operon.

The present disclosure provides for the use of said 4-acetoxy-2-methylene-butan-1-ol obtained by whole cell biotransformation as defined herein above and in the appended claims for the synthesis of α-methylene-γ-butyrolactone (MBL). The present disclosure also provides for the synthesis of 2-methylenebutan-1,2-diol by the hydrolysis of 4-acetoxy-2-methylene-butan-1-ol, wherein said 4-acetoxy-2-methylene-butan-1-ol is obtained by whole cell biotransformation as defined herein above and in the appended claims. Said hydrolysis may be under basic conditions, under acidic conditions or enzymatically using an appropriate lipase or carboxyl esterase enzyme.

The present disclosure still further provides a process for preparing a compound of Formula (BIII) from a compound of Formula (BII) by whole cell biotransformation:

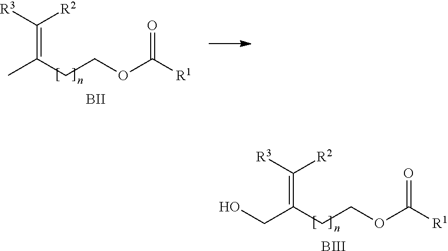

wherein:
n is an integer of from 0 to 8;
R¹ is C₁-C₄ alkyl;
R² is H or C₁-C₄ alkyl; and,
R³ is H or C₁-C₄ alkyl,
   said process comprising:
i) contacting a cell (CB) with a culture medium containing said compound of Formula (BII) or with a culture medium contiguous with an organic phase containing said compound of Formula (BII) under conditions that enable the cell to form said compound of Formula (BIII) from said compound of Formula (BII); and,
ii) isolating the resultant compound of Formula (BIII), wherein said cell (CB) exhibits activity of at least one alkane monooxygenase enzyme which catalyzes the Cₙ₊₃-hydroxylation of said compound of Formula (BII).

The utilized cell (CB) of this aspect of the disclosure possesses the gene of an alkane monoxygenase, optionally as part of an Alk operon.

### Definitions

As used herein, the singular forms “a”, “an” and “the” include plural referents unless the context clearly dictates otherwise.

The terms “comprising”, “comprises” and “comprised of” as used herein are synonymous with “including”, “includes”, “containing” or “contains”, and are inclusive or open-ended and do not exclude additional, non-recited members, elements or method steps.

As used herein, the term “consisting of” excludes any element, ingredient, member or method step not specified. For completeness, the term “comprising” encompasses “consisting of”.

When amounts, concentrations, dimensions and other parameters are expressed in the form of a range, a preferable range, an upper limit value, a lower limit value or preferable upper and limit values, it should be understood that any ranges obtainable by combining any upper limit or preferable value with any lower limit or preferable value are also specifically disclosed, irrespective of whether the obtained ranges are clearly mentioned in the context.

Further, in accordance with standard understanding, a weight range represented as being “from 0” specifically includes 0 wt. %: the ingredient defined by said range may or may not be present in the composition.

The words “preferred”, “preferably”, “desirably” and “particularly” are used frequently herein to refer to embodiments of the disclosure that may afford particular benefits, under certain circumstances. However, the recitation of one or more preferable, preferred, desirable or particular embodiments does not imply that other embodiments are not useful and is not intended to exclude those other embodiments from the scope of the disclosure.

As used throughout this application, the word “may” is used in a permissive sense—that is meaning to have the potential to—rather than in the mandatory sense.

As used herein, room temperature is 23° C. plus or minus 2° C. As used herein, “ambient conditions” means the temperature and pressure of the surroundings in which the composition is located or in which an adhesive or coating obtained from said composition is located.

The term “isoprenol” refers to compounds of Formula C₅H₁₀O. The IUPAC name of isoprenol is 3-methylbut-3-en-1-ol and synonyms of isoprenol include isobutenylcarbinol and methallyl carbinol.

For completeness, “Tulipalin A” has been referred to herein interchangeably with α-methylene-γ-butyrolactone. The IUPAC name of Tulipalin A is 3-methylideneoxolan-2-one (C₅H₆O₂).

The term “acetylating” or “acetylation” as used herein, refers to the process of introducing an acetyl group into the molecule of a compound having hydroxyl (—OH) groups. The acetylation replaces H of said —OH groups with CH₃CO-groups.

As used herein, “C₁-Cₙ alkyl” group refers to a monovalent group that contains 1 to n carbons atoms, that is a radical of an alkane and includes straight-chain and branched organic groups. As such, a “C₁-C₄ alkyl” group refers to a monovalent group that contains from 1 to 4 carbons atoms, that is a radical of an alkane and includes straight-chain and branched organic groups. Examples of alkyl groups include, but are not limited to: methyl; ethyl; propyl; isopropyl; n-butyl; isobutyl; sec-butyl; and, tert-butyl. In the present invention, such alkyl groups may be unsubstituted or may be substituted with one or more halogen. Where applicable for a given moiety (R), a tolerance for one or more non-halogen substituents within an alkyl group will be noted in the specification.

As used herein, the term “water” is intended to encompass tap water, spring water, purified water, de-ionized water, de-mineralized and distilled water. Water is included in the compositions of the present invention in its liquid form. The presence of solid water particles—ice—is not desirable as solid water cannot be mobilized for the formation of the hydrates required for the development of strength in cured composition.

As used herein “basicity” means the quality of being a base, not an acid. More particularly, in accordance with the Lewis theory of acids and bases, a base is an electron-pair donor. This definition encompasses but is not limited to Brønsted-Lowry bases, which compounds act as proton acceptors.

As used herein, the terms “monome” and “co-monome” refer to a molecule that is capable of conversion to polymers, synthetic resins or elastomers by combination with itself or other similar molecules or compounds. The terms are not limited to small molecules but include oligomers, polymers and other large molecules capable of combining with themselves or other similar molecules or compounds.

As used herein, “macro-monomer” refers to a polymer having at least one functional group through which polymerization reactions can proceed. Macro-monomers are thus macromolecular monomers which can be converted to homo- or copolymers of defined structures. It is not precluded that a macro-monomer as used herein comprises more than one polymeric chain attached to one functional group.

As used herein, the term “enzyme” refers to a protein that catalyzes a chemical reaction. The catalytic function of an enzyme constitutes its “enzymatic activity” or “activity”. As used herein, the term “substrate” refers to a substance on which an enzyme performs its catalytic activity to generate a product.

As used herein, the term “monooxygenase” or “hydroxylase” refers to an enzyme as defined in BRENDA:EC (Enzyme Commission) 1.14 that catalyzes the incorporation of on hydroxyl group into a substrate such as an alkane or alkene requiring electron donors such electron transfer proteins, NADH or NADPH.

As used herein, a “laccase” is a multi-copper containing oxidase as defined in BRENDA:EC (Enzyme Commission) 1.10.3.2 that catalyzes the oxidation of alcohols by single-electron abstraction, with the concomitant reduction of oxygen to water in a four-electron transfer process. includes zinc enzymes requiring NAD+ or NADP+ as an acceptor as defined in BRENDA:EC1.1.1.1 and metal-independent enzymes requiring NAD+ or NADP+ as an acceptor as defined in BRENDA:EC1.1.1.3.

As used herein an “aldehyde dehydrogenase” (AlDH) is an enzyme requiring NAD+ or NADP+ as an acceptor as defined in BRENDA:EC1.2.1.3. As used herein, the term “alcohol oxidase” (AlcOx) references as oxidoreductase enzyme which utilizes oxygen as an acceptor, as defined in BRENDA:EC1.1.3.13.

As used herein, the terms “mediating compound” or “mediator” may be used interchangeably to refer to a chemical compound that functions as a redox mediator to shuttle electrons between an enzyme exhibiting oxidizing activity and a secondary substrate or electron donor. Such chemical mediating compounds may be referenced in the art as “enhancers” and “accelerators”.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The parameter “nucleotide identity” relative to a recorded sequence listing (Sequence ID Number) may be determined herein using established known methods. Mention may be made of the Smith-Waterman algorithm computer programs running this and equivalent algorithms include but are not limited to: GCG Software, including GAP as described in Deveroy, J. et al., Nucleic Acid Research 12: 387, 1984; and, BLASTP, BLASTN and FASTA as described in inter alia Altschul, S. et al., Journal of Molecular Biology 215: 403-410, 1990.

As used herein, the term “expression vector” refers to a DNA construct containing a DNA coding sequence (e.g., gene sequence) that is operably linked to one or more suitable control sequence(s) capable of effecting expression of the coding sequence in a host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle or a potential genomic insert. Once transformed into a suitable host, the vector may replicate and function independently of the host genome, or may, in some instances, integrate into the genome itself.

The term “whole cell biotransformation” refers to a conversion of a suitable substrate to a product by a microorganism.

The term “wild type” as used herein with reference to a cell denotes a cell with a genomic make-up that is in a form as found naturally in the wild. The term “wild type” does not therefore include such cells or such genes where the gene sequences have been altered at least partially by man by deliberate mutation.

As used herein with respect to proteins, the term “mutant” refers to a version of a protein where the version is other than wild-type. The change may be affected by methods well known to one skilled in the art, for example, by point mutation in which the resulting protein may be referred to as a mutant.

As used herein the term “non-recombinant laboratory cell” refers to a cell obtained from a cell line which is a clonal population derived from a progenitor wild type cell. The clonal population is often obtained by continuous or prolonged growth and division in vitro. Spontaneous or induced changes can occur in karotype during laboratory storage or transfer of clonal populations: as such, the laboratory cell derived from the cell line may not be precisely identical to the ancestral cell and the cell line encompasses those variants.

Herein the term “operon” denotes a functioning unit of DNA containing a cluster of genes under the control of a single promoter.

Optical density at a wavelength of 600 nm, abbreviated to “OD₆₀₀” refers to a standard measure of cell density in a culture obtained by calculating the absorbance of a 1 cm pathlength of culture of light having a 600 nm wavelength minus the absorbance of a 1 cm pathlength of the medium without the cell culture.

The term “medium” as used in reference to a cell culture includes the components of the environment surrounding the cells. Media may be solid, liquid, gaseous or a mixture of phases. Media include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. The term “medium” also refers to material that is intended for use in a cell culture, even if it has not yet been contacted with cells.

The term “minimal medium” includes media that support growth of the wild-type form of a species but do not support growth of one or more auxotrophic strains of that species. A supplemented minimal medium is a minimal medium that includes one or more additional substances in order to support growth of an auxotrophic strain. “Defined medium” or “defined minimal medium” refer to media that are made of chemically defined (usually purified) components. “Defined media” do not contain poorly characterized biological extracts such as yeast extract and beef broth.

For completeness, a medium suitable for growth of a high-density culture is any medium that allows a cell culture to reach an OD₆₀₀ of 3 or greater when other conditions—such as temperature and oxygen transfer rate—permit such growth.

Viscosities of the compositions described herein are, unless otherwise stipulated, measured using the Brookfield Viscometer at standard conditions of 20° C. and 50% Relative Humidity (RH). The method of calibration, the spindle type and rotation speed of the Brookfield Viscometer are chosen according to the instructions of the manufacturer as appropriate for the composition to be measured.

As used herein, “polymerization conditions” are those conditions that cause the at least one monomer to form a polymer, such as temperature, pressure, atmosphere, ratio of starting components used in the polymerization mixture, reaction time, or external stimuli of the polymerization mixture. The polymerization process can be carried out in bulk, or solution, or other conventional polymerization modes. The process is operated at any of the reaction conditions appropriate to the polymerization mechanism.

The term “anionic polymerization” as used herein refers to the ionic polymerization mechanism in which the kinetic chain carriers are anions. Accordingly, an anionic polymerization reaction is a chain reaction in which the growth of the polymer chain proceeds by reaction(s) between the monomer(s) and the reactive site(s) on the polymer chain with regeneration of the reactive site(s) at the end of each growth step. Herein the anionic polymerization is used to produce macromolecules from monomers that contain a carbon-carbon double bond. The polymerizations are initiated by nucleophilic addition to the double bond of the monomer, wherein the initiator comprises an anion, such as hydroxide, alkoxides, cyanide, or a carbanion.

As used herein, the term “ring-opening polymerization” denotes a polymerization in which a cyclic compound (monomer) is opened to form a linear polymer in the presence of an appropriate catalyst. The reaction system tends towards an equilibrium between the desired resulting high-molecular compounds, a mixture of cyclic compounds and/or linear oligomers, the attainment of which equilibrium largely depends on the nature and amount of the cyclic monomers, the catalyst used and on the reaction temperature. The use of solvents and/or emulsions in the polymerization is not recommended as their removal once the reaction is complete can be complex.

The present compositions may be defined herein as being “substantially free” of certain compounds, elements, ions or other like components. The term “substantially free” is intended to mean that the compound, element, ion or other like component is not deliberately added to the composition and is present, at most, in only trace amounts which will have no (adverse) effect on the desired properties of the composition. An exemplary trace amount is less than 1000 ppm by weight of the composition. The term “substantially free” encompasses those embodiments where the specified compound, element, ion, or other like component is completely absent from the composition or is not present in any amount measurable by techniques generally used in the art.

The term “anhydrous” as used herein has equivalence to the term “substantially free of water” Water is not deliberately added to a given composition and is present, at most, in only trace amounts which will have no (adverse) effect on the desired properties of the composition.

### DETAILED DESCRIPTION OF THE INVENTION

The isoprenol provided to step a) of the present disclosure may be sourced commercially and, as such, could be derived from petroleum-based isobutene and formaldehyde. However, it is preferred that the compound be sourced from those synthetic microbial systems for the production of isoprenol which are known in the art.

In accordance with an important embodiment of the present disclosure said isoprenol of step a) is obtained via a fermentation stage, said stage comprising:
providing a fermentation medium comprising a substrate for microbial growth; and,
introducing into said medium an inoculant comprising a culture of one or more microorganisms selected from the group consisting of bacteria, moulds and yeasts, wherein said one or more microorganisms is characterized in that it ferments said carbohydrate to form isoprenol.

The term “fermentation medium” is used herein to denote a three phase (solid-liquid-gas) system which is retained within the fermentation vessel. The liquid phase contains water, dissolved nutrients, dissolved substrates for microbial growth and dissolved metabolites; the source of the water is not limited and includes, in particular, process waters, such as backset and/or thin stillage, scrubber water, evaporator condensate or distillate, side stripper water from distillation, or other fermentation product plant process water. The solid phase comprises individual cells, pellets, insoluble substrates for microbial growth and precipitated metabolic products.

In the context of microbial growth, the term “substrate” refers to any substance or compound that is converted or meant to be converted into another compound by the action of an enzyme. The term “substrate” is intended to encompass not only compounds that provide a carbon source suitable for use as a starting material, such as any biomass derived carbohydrate, but also intermediate metabolites used in a metabolic pathway associated with a microorganism. The fermentation medium may typically comprise, as a substrate, one or more fermentable carbohydrates, such as sugars.

The fermentation medium, including fermentation substrate and other raw materials used in the fermentation process of the invention may be processed—by milling, liquefaction, saccharification or the like—prior to or simultaneously with the fermentation process. Accordingly, the fermentation medium can refer to the medium before the fermenting micro-organism is added, such as, the medium in or resulting from liquefaction and/or saccharification, as well as the medium which comprises the fermenting organism, such as, the medium used in simultaneous saccharification and fermentation (SSF) or one-step fermentation processes. For completeness, in that embodiment mentioned above where the antibacterial agent is added to the fermentation medium before the inoculating micro-organism, this includes the addition of said agent during liquefaction and/or saccharification.

As used herein “inoculant” means the original source of a complex microbial community which is intended to be added to the fermentation vessel but which does not limit the final composition of the microbial community; the final composition is determined by the operating conditions and productivity of the fermentation vessel. The inoculant is typically formed by propagation of the desired microorganism(s) in a suitable propagation tank, which tank is much smaller than the fermentation vessel.

The inoculant typically includes a culture of one or more production strains of microorganisms which may have been adapted by natural selection or by biotechnological means to produce the fermentation product of interest. An exemplary, but non-limiting, inoculant contains a culture of Escherichia coli as described in Kang et al. Isopentenyl diphosphate (IPP)-bypass mevalonate pathways for isopentenol production Metabolic Engineering Vol. 34: 25-25 (2016).

Further instructive references for the fermentive production of isoprenol are known. For example, US 20080092829 (Renninger et al.) describes the production of isoprenol from microorganisms, including bioengineered microorganisms: one disclosed biosynthetic route involves the mevalonate (MVA) pathway wherein mevalonate is produced, diphoshorylated, then decarboxylated-dehydrated into isoprenyl-pyrophosphate, and finally dephosphorylated twice into isoprenol. The formation of isoprenol from E. coli strains via the 2-methyl-D-erythritol-4-phosphatase (MEP) pathway is also known. Further, EP 2 516 656 A (Global Bioenergies et al.) discloses a method for the production of isoprenol characterized in that it comprises the step of converting mevalonate with an enzymatic pathway involving an enzyme having the activity of a diphosphomevalonate decarboxylase (EC 4.1.1.33) into isoprenol.

The importance of this fermentation embodiment is that it enables the coupling of a fermentation process yielding isoprenol from a carbohydrate source to the steps a) to c) of the present disclosure, which steps may each be performed by whole-cell biotransformation. More particularly, as described hereinbelow, a singular production strain of a microorganism—such as, but not limited to, E. coli—may be used to yield 4-acetoxy-2-methylene butyric acid starting from a carbohydrate source, provided said production strain exhibits the enzyme activities by which the step b) cell (CB) and the step c) cell (CC) are characterized.

### Step A)

Step a) of the present disclosure provides for the O-protection of the C1-hydroxyl group of isoprenol via acetylation:

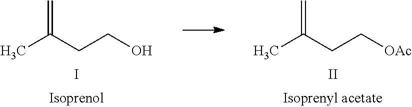

The acetylation reaction of step a) may simply comprise the treatment of isoprenol with an acetylation reagent under effective acetylation conditions. Non-limiting examples of suitable acetylation reagents include acetic acid, acetic anhydride and acetyl chloride: the use of more than one of said reagents is not precluded. Further, the acetylation conditions may include a temperature of from 20 to 150° C., for example from 20 to 100° C. The use of a catalyst may be beneficial and, without intention to limit the present disclosure, mention in this regard may be made of: perchloric acid; magnesium perchlorate; copper perchlorate; sulphuric acid; hydrochloric acid; hydrobromic acid; trichloroacetic acid; copper chloride; copper sulphate; zinc chloride; zinc acetate; zinc sulphate; meta potassium periodate; dimethyl sulphate; and, diethyl sulphate.

The above aside, it is not precluded that step a) of the present disclosure be constituted by an enzymatic process performed either in vitro or via whole cell biotransformation. In the latter regard, US20100180491A1 (Taek-Soon Lee et al.) describes the culturing of a genetically modified host cell which expresses an enzyme capable of catalyzing the esterification of isoprenol and inter alia acetic acid, such as an alcohol acetyltransferase (AAT), wax ester synthase/diacylglycerol acyltransferase (WS/DGAT) or lipase, under a suitable condition so that isoprenyl acetate is produced. Further, Zada et al. Metabolic engineering of Escherichia coli for production of mixed isoprenoid alcohols and their derivatives, Biotechnology for Biofuels, Volume 11: 210 (2018) also describes the acetylation of the C1-hydroxy group of isoprenol: therein acetyl coenzyme A (Acetyl-CoA) acts as the acetylation reagent under the enzymatic catalysis of an acetyl transferase, specifically chloramphenicol acetyltransferase (CAT).

Irrespective of the chemical or biochemical mode of acetylation, the progress of the above reaction can be monitored by known techniques of which mention may be made of ¹H NMR, Fourier Transform Infrared Spectroscopy, Ultra Performance Liquid Chromatography (UPLC), gas chromatography or thin layer chromatography (TLC). At an appropriate conversion in the chemical acetylation process, an aqueous base—such as sodium or potassium hydrogen carbonate—may be added to quench the reaction.

The reaction product (II) may be isolated and may be used in step b) in crude form. Alternatively, the isoprenyl acetate (II) may be purified using methods known in the art including, but not limited to, solvent extraction, filtration, evaporation and chromatography.

### Step B)

This step of the present disclosure provides for the formation of 4-acetoxy-2-methylene-butan-1-ol (III) by whole cell biotransformation. This is depicted in the following reaction scheme:

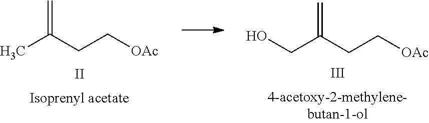

The utilized cell (CB) of this step possesses the gene of an alkane monoxygenase, optionally as part of an Alk operon. In an embodiment, the utilized cell (CB) may be a wild type cell or non-recombinant, laboratory cell which possesses the gene of an alkane monoxygenase, optionally as part of an Alk operon, in particular an Alk operon containing the gene products AlkB, AlkH and AlkJ. Mention may be made of the Pseudomonas putida, the wild type genotype of which contains two Alk operons: the first operon encodes the gene products AlkB, AlkF, AlkG, AlkH, AlkJ, AlkK and AlkL; the second operon encodes AlkS and AlkT, wherein AlkS has a regulatory function on the expression of the first alk operon.

In the alternative, the utilized cell (CB) may have been genetically modified relative to its wild type so that, in comparison with said wild type, it is able to produce more 4-acetoxy-2-methylene-butan-1-ol (III) starting from isoprenyl acetate (II). This comparison is intended to encompass both: i) that case where the wild type of the genetically modified cell produces detectable amounts of 4-acetoxy-2-methylene-butan-1-ol; and, ii) that case where the wild type of the genetically modified cell is not able to form any detectable amount of 4-acetoxy-2-methylene-butan-1-ol. Thus, as regards said second category of cells ii), it is only following the genetic modification of the wild type to produce the cell used in step b) that a detectable amount of 4-acetoxy-2-methylene-butan-1-ol is formed.

It is preferred for the utilized cell (CB) to have been genetically modified so that, in a defined time interval of 24 hours, it forms at least 10 times, for example at least 100 times or at least 1000 times, more 4-acetoxy-2-methylene-butan-1-ol (III) than the wild-type cell. The increase in product formation may be determined by separately cultivating the cell used according to step b) of the present disclosure and the wild-type cell under the same initial cell density, nutrient medium and culture conditions for the specified time interval and then determining the amount of the target product in each nutrient medium.

The cells (CB) used in step b) of the present disclosure may be prokaryotes or eukaryotes. They may be mammalian cells—including human cells—plant cells or microorganisms, such as fungi, molds or bacteria, wherein preferred microorganisms are those that have been deposited with Deutsche Sammlung von Mikroorganismen und Zellkulturen GmbH (DSMZ). Whilst yeasts may be important microorganisms (fungi) in this context, it is preferred to use bacteria and an instructive reference may therefore be made to http://www.dsmz.de/species/bacteria.htm.

The potential use of wild type Pseudomonas putida in the present invention has been noted above. Without intention to limit the present invention, preferred cells (CB) that may be genetically modified and used in the step b) of the present disclosure may be selected from the genera: Corynebacterium; Brevibacterium; Bacillus; Lactobacillus; Lactococcus; Candida; Pichia; Kluveromyces; Saccharomyces; Escherichia; Zymomonas; Yarrowia; Methylobacterium; Ralstonia; Pseudomonas; Burkholderia; and, Clostridium. A particular preference may be mentioned for the use of cells (CB) selected from the group consisting of Escherichia coli, Corynebacterium glutamicum and Pseudomonas putida. Good results have been obtained with the E. coli BL21 strain.

In comparison with its wild type, a genetically modified cell (CB) according to step b) must exhibit increased activity of at least one of alkane monooxygenase enzyme which catalyzes the C4-hydroxylation of isoprenyl acetate: the direct C4-hydroxylation of isoprenyl acetate (II) leads to 4-acetoxy-2-methylene-butan-1-ol (III).

The enzyme alkane monooxygenase may be encoded by the AlkB gene from Pseudomonas putida GP01, mutants and homologues thereof. For example, the enzyme alkane monooxygenase may be encoded by the AlkB gene from Pseudomonas putida GP01 selected from the group consisting of: the AlkBGT gene cluster; the AlkBGTJH gene cluster; and, the AlkBGTJHL gene cluster. The isolation of the respective gene sequence is described for example in van Beilen et al., “Functional Analysis of Alkane Hydroxylases from Gram-Negative and Gram-Positive Bacteria”, Journal of Bacteriology, Vol. 184 (6), pages 1733-1742 (2002). Moreover, the DNA sequence information encoding AlkB, AlkF, AlkG and AlkT were obtained from the Pseudomonas putida OCT Plasmid alk gene cluster identified as GenBank AJ 245436.1; and, the plasmid sequence for the pCom10 vector into which AlkBGT is cloned can be found in GenBank AJ 302087.1.

It is considered that mutants may provide for improvement of the activity of AlkB towards isoprenyl acetate and may be determined by rational design based on a predictive model. For example, the structure of AlkB may be predicted by the AlphaFold2 predictive software available from European Molecular Biology Laboratory-European Bioinformatics Institute (EMBL-EBI); positions are elected from mutation based on docking studies using isoprenyl acetate as a ligand. Single mutants of each selected position may be generated by site-directed mutagenesis of the pBT10 construct. Exemplary mutants include but are not limited to: AlkB(I233V), mutant disclosed in Koch et al. [8]; and, AlkB(F164L), a single mutant identified from docking studies using isoprenyl acetate as ligand in which the amino acid phenylalanine (Phe) of the wild-type AlkB is exchanged to leucine (Leu).

Enzymes that are encoded by nucleic acid sequences that have at least 40%, preferably at least 50% and more preferably at least 75% identity to the identified sequences are suitable for use in the method of the present invention. For completeness, further exemplary homologues of the AlkB gene include but are not limited to: AlkB-P1 obtained from Pseudomonas putida P1; Alk1-MO obtained from Marinobacter hydrocarbonoclasticus; and, AlkB1 obtained from Alcanivorax borkumensis.

To achieve an increased intracellular activity of the aforementioned enzymes in the genetically modified cells (CB), one or more of the following measures may be employed: an increase of the copy number of the gene sequence(s) that code for the enzyme; the use of a strong promoter for the gene; the use of a stronger ribosome binding site; the use of codon optimization; the employment of a gene or allele that codes for a corresponding enzyme with increased activity; and, the employment of an altered amino acid sequence of the above mentioned enzymes, exhibiting increased activity, as described, for instance, in Koch et al., “In Vivo Evolution of Butane Oxidation by Terminal Alkane Hydroxylases AlkB and CYP153A6”, Applied and Environmental Microbiology, Vol. 75 (2), pages 337-344 (2009).

Genetically modified cells used in the method according to the invention are produced by transformation, transduction and/or conjugation with an expression vector that contains the desired gene, an allele of this gene or parts thereof and a vector that makes expression of the gene possible. Heterologous expression is achieved through the integration of the gene or the alleles thereof in the chromosome of the cell or an extra-chromosomally replicating vector. Without intention to limit the present disclosure, WO2009/077461 (Evonik Degussa GmbH) provides an instructive reference of cellular genetic transformation.

In the production of 4-acetoxy-2-methylene-butan-1-ol, the following sub-steps may be identified:
b) i) contacting the cell (CB) as previously defined either with a culture medium containing isoprenyl acetate or with a culture medium contiguous with an organic phase containing isoprenyl acetate under conditions that enable the cell to form 4-acetoxy-2-methylene-butan-1-ol from isoprenyl acetate; and,
optionally b) ii) isolation of the resultant 4-acetoxy-2-methylene-butan-1-ol.

The cells (CB) can be brought into contact with said culture medium in a bioreactor, and therefore cultivated, either continuously or discontinuously in a batch process, a fed-batch process or a repeated-fed-batch process.

A conventional batch bioreactor system will often be considered “closed” where the composition of the medium is established at the beginning of a respective bio-production event and not subject to artificial alterations and additions during the time period ending substantially with the end of the bio-production event. In this circumstance, at the beginning of the bio-production event, the medium is inoculated with the desired microorganism(s) and bio-production is permitted to occur without adding anything to the system. In the present disclosure, however, the “batch” type of bio-production event encompasses those systems in which the substrate content of the medium is established at the beginning of the respective bio-production event but attempts are made at controlling factors such as pH and oxygen concentration.

A variation on the standard batch system is the fed-batch system. Fed-batch bio-production processes comprise a typical batch system with the exception that the nutrients, including the substrate, are added in increments as the bio-production progresses. Fed-Batch systems are useful when catabolite repression is apt to inhibit the metabolism of the cells and where it is desirable to have limited amounts of substrate in the media. Measurement of the actual nutrient concentration in Fed-Batch systems may be made directly, by sampling for example, or estimated on the basis of the changes of measurable factors such as pH, dissolved oxygen and the partial pressure of waste gases such as CO₂.

Continuous bio-production is considered an “open” system where a defined bio-production medium is added continuously to a bioreactor and an equal amount of conditioned media is removed simultaneously for further processing. Continuous bio-production, whether in a chemostat or perfusion culture system, generally maintains the cultures within a controlled density range, wherein cells are primarily in log phase growth. Continuous bio-production can limit the down time associated with draining, cleaning and preparing the equipment for the next bio-production event. Furthermore, it tends to be more economical to continuously operate downstream unit operations, such as distillation, than to run them in batch mode. And continuous bio-production permits the modulation of one or a plurality of factors that affect cell growth or end product concentration.

Within the bioreactor, the bio-production system is maintained within a suitable temperature range and within a controlled dissolved-oxygen concentration range for a sufficient time to obtain the desired conversion (C4-hydroxylation) of the isoprenyl acetate (II) substrate molecules to yield the chemical product (III). A temperature range of from 20° C. to 50° C., for example from 20° C. to 40° C., may be mentioned as being suitable for this step. Anaerobic conditions may be maintained in the bioreactors used in this step but it is more typical to maintain aerobic conditions therein via the addition of oxygen or an oxygen containing gas, such as air. The dissolved oxygen levels of a liquid culture comprising a nutrient media may be monitored to maintain or confirm a desired aerobic, microaerobic or anaerobic condition.

It will be evident to the skilled artisan that the culture medium used in this step must be suitable for the requirements of the microorganism(s) used to produce the desired end product (III). To form the culture medium, an appropriate combination may be made of: at least one growth factor or a precursor thereof; at least one nitrogen source; at least one phosphorous source; and, at least one source of trace metal selected from the group consisting of manganese, boron, cobalt, copper, molybdenum, zinc, calcium, magnesium, iron, nickel and combinations thereof. Suitable growth factors include but are not limited to amino acids and vitamins, such as biotin, vitamin B12, derivatives of vitamin B12, thiamin and pantothenate. Nitrogen-sources include but are not limited to peptones, yeast extract, meat extract, malt extract, corn-steep liquor, soybean flour, ammonium sulphate, ammonium chloride, ammonium phosphate, ammonium carbonate and ammonium nitrate. Phosphorous sources include but are not limited to phosphoric acid, sodium dihydrogen phosphate, potassium dihydrogen phosphate, disodium hydrogen phosphate or dipotassium hydrogen phosphate. It will further be recognized that the medium may contain adjuvants such as: pH adjuster regulators including mineral acids and bases, for instance sodium hydroxide, potassium hydroxide, ammonia, ammonia water; antifoaming agents; and, antibiotics to maintain plasmid stability.

The medium may be a defined medium. It is also noted that step b) may be performed utilizing a minimal medium or a supplemented minimal medium. Further, the use of commercially prepared media is not precluded and particular mention may be made of: Luria Bertani (LB) broth; M9 Minimal Media; Sabouraud Dextrose (SD) broth; Yeast Medium (YM) broth, and Yeast Synthetic Minimal Media (Ymin).

In the aforementioned optional sub-step b) ii), the resultant 4-acetoxy-2-methylene-butan-1-ol may be isolated from the bioreactor. This sub-step is regarded as optional as the present invention encompasses an embodiment wherein steps b) and c) are both performed by whole cell biotransformation and wherein the cells utilized in each step (CB, CC below) are the same. That embodiment aside, dependent on the cell (CB) used, the isolation of 4-acetoxy-2-methylene-butan-1-ol may either be from the culture medium if the host cell secretes it into the medium or directly from the host cell producing it, if it is not so-secreted. The isolated compound (III) may optionally be purified using methods known in the art including, but not limited to, solvent extraction, filtration, evaporation, distillation, crystallization and chromatography.

### Step C)

This step of the present disclosure provides for the oxidation of the 4-acetoxy-2-methylene butan-1-ol (III) to 4-acetoxy-2-methylene butyric acid (V). This oxidation may be viewed as a two-stage process (i), ii)) in which 4-acetoxy-2-methylene-butanal (IV) is formed as an intermediate product, as given in the following reaction scheme.

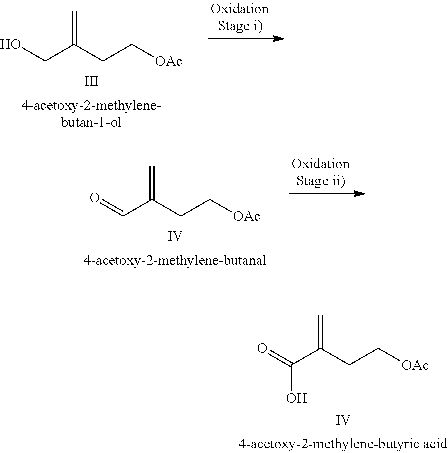

Step c) of the present process may broadly be separated into two main embodiments: in a first embodiment, the two oxidation stages (c)i), c)ii)) are distinct and separate with intermediate isolation and, optionally, purification of the 2-acetoxy-2-methylene-butanal (IV); and, in a second embodiment, the two oxidation stages proceed without this intermediate isolation of 2-acetoxy-2-methylene-butanal (IV) and thus as a “one-pot” synthesis. The performance of the second embodiment does not however preclude the reaction conditions being modified over time to sequentially drive each oxidation stage.

It is considered that this reaction sequence may either be realized by chemical oxidation via the use of oxidizing reagents, by biocatalytic oxidation or by whole cell biotransformation either within a whole-cell biocatalyst that catalyzes both steps b) and c) or within separated whole-cell biocatalysts that catalyze step c) separately from step b). The use of either biocatalytic oxidation or whole cell biotransformation is preferred herein. Traditional chemical oxidation methods often require the use of at least equimolar amounts of oxidizing reagents, whose atom efficiency is relatively low. Moreover, the practical application of chemical routes is often limited by overoxidation, poor selectivity and the use of organic solvents. Conversely, biocatalytic oxidation and whole cell biotransformation are characterized by excellent selectivity as well as mild reaction conditions.

### Step c) Chemical Oxidation

Where step c) is performed chemically in two distinct reaction stages, the first stage thereof (c)i)) is the oxidation of the primary alcohol (III) to the corresponding aldehyde (IV). There is no particular intention to limit the oxidizing agents which may find utility in this stage. Exemplary reagents include: oxides of copper, cobalt and chromium; copper, silver and mixtures thereof; platinum; platinum dioxide; ceric ammonium nitrate; sodium bromate; lead tetraacetate; hexavalent chromium; sodium or potassium dichromate; pyridinium dichromate; chromic acid; Collins reagent; pyridinium chlorochromate; chromyl chloride; di-tert-butyl chromate; manganese dioxide; tetrachloro-o-benzoquinone; tetrachloro-p-benzoquinone; 2,3-dichloro-5,6-dicyano-p-benzoquinone; dimethyl sulfide and chlorine; hypervalent iodine compounds; N-chlorosuccinimide (NCS); tert-butyl hydroperoxide (TBHP); and, dimethylsulfoxide (DMSO). U.S. Pat. No. 5,132,465 A provides an instructive background reference in this regard.

The second stage (c)ii)) entails the oxidation of the aldehyde (IV) to the corresponding carboxylic acid (V). Suitable oxidizing agents for this step include but are not limited to: Jones' reagent; potassium permanganate; silver oxide; and, nitric acid. Such oxidizing agents are used together with a catalyst, such as sulfuric acid, and it is moreover desirable to use either water as a solvent or a water-miscible solvent such as acetone, tetrahydrofuran, methanol or ethanol. The oxidizing reaction (stage c)ii)) is conventionally performed at a temperature of from 20 to 50° C. for a period of from 0.5 to 5 hours.

As will be recognized by the skilled artisan, the direct or “one-pot” oxidation of 4-acetoxy-2-methylene butan-1-ol (III) to 4-acetoxy-2-methylene butyric acid (IV) can be effected under certain circumstances: typically a stoichiometric excess of the oxidizing agent will be required and the aldehyde (IV) formed as the intermediate product must be retained in the reaction mixture. In aqueous media, 4-acetoxy-2-methylene butyric acid (V) will represent the major product when using a stochiometric excess of an oxidizing agent selected from: chromic acid (H₂CrO₄); Na₂CrO₄; K₂CrO₄; K₂Cr₂O₇; K₂Cr₂O₇; permanganate; and, Jones' reagent. For completeness, a stoichiometric excess of said Jones' reagent means a stoichiometric excess of the chromic acid thereof relative to the 4-acetoxy-2-methylene butan-1-ol (III). Alternative methods which may be suitable for oxidizing 4-acetoxy-2-methylene butan-1-ol (III) to 4-acetoxy-2-methylene butyric acid (V) without the intermediate isolation of 2-acetoxy-2-methylene-butanal (IV) are disclosed in inter alia: WO99/52849; WO2006/001387; and, US2009/0124806A1.

The progress of the reaction(s) of step c) can be monitored by known techniques of which mention may be made of ¹H NMR, Fourier Transform Infrared Spectroscopy, Ultra Performance Liquid Chromatography (UPLC), gas chromatography or thin layer chromatography (TLC).

The 4-acetoxy-2-methylene butyric acid (V) product of step c) may be isolated from the reactor. Prior to its use in subsequent synthesis steps, the isolated 4-acetoxy-2-methylene butyric acid (V) may optionally be purified using methods known in the art including, but not limited to, solvent extraction, filtration, evaporation, distillation, crystallization and chromatography.

### Step c) Enzymatic Oxidation

As regards biocatalytic oxidation, it is not prudent to propose an overriding preference for the use of either enzymatic oxidation or whole cell biotransformation based on genetically modified cells. Certainly, when compared with isolated enzymes, the use of whole-cell catalysts simplifies the procedure, reduces costs and improves the enzyme stability. However, in certain circumstances, the active form of the enzyme will preclude its use in whole cell biotransformation.

The present disclosure provides for step c) being characterized by an enzymatic oxidation process comprising contacting 4-acetoxy-2-methylene butan-1-ol (III) under aerobic conditions with at least one enzyme exhibiting oxidizing activity and, optionally, at least one mediating compound which enhances the oxidizing activity of the enzyme. The reaction mixture should preferably comprise from 0.001 to 10 mg of said at least one enzyme per kg of 4-acetoxy-2-methylene butan-1-ol (III).

Said at least one enzyme exhibiting oxidizing activity will conventionally be selected from alcohol and aldehyde oxidizing enzymes: such enzymes with a broad substrate spectrum are described in the literature and mention may be made of: alcohol dehydrogenases (ADH) such as Escherichia coli EcAdhZ3-LND; alcohol oxidases (AlcOx) such as Aspergillus fumigatus LCAO_Af; aldehyde dehydrogenases (AlDH); and, laccase.

Without intention to limit the present invention, a useful alcohol dehydrogenase (ADH) enzyme may be encoded by a homologue of the AlkJ gene and a useful aldehyde dehyrogenase (AlDH) enzyme may be encoded by the AlkH gene from Pseudomonas putida GP01. The DNA sequence information for AlkJ alcohol dehydrogenase and the AlkH dehydrogenase can be taken from the Pseudomonas putida OCT Plasmid alk gene cluster identified as GenBank AJ 245436.1. And enzymes that are encoded by nucleic acids that have at least 40%, preferably at least 75% and more preferably at least 90% identity to the recorded sequences are suitable for use in the method of the present invention.

As will be recognized by the skilled artisan, an operable activity of alcohol dehydrogenase (ADH) enzymes might only be achieved in the presence of oxidized cofactors, in particular oxidized nicotinamide cofactors, as hydride acceptors. Similarly, alcohol oxidases (AlcOx) which function by transferring reducing equivalents to molecular oxygen, generating H₂O₂ as a stoichiometric by-product, can be flavin-dependent or Cu²⁺ dependent. Furthermore, it is noted that ADHs and AlcOxs are generally not capable of oxidizing aldehydes because the aldehyde proton is not abstractable as a hydride: this mechanistic limitation can be solved by providing a nucleophilic cofactor which attacks the carbonyl group transiently turning it into an alcohol containing a hydridically abstractable proton. Aldehyde dehydrogenases (AlDH) utilizes this approach via a cysteine moiety in the enzyme active site.

Based on the above considerations, it will be conventional for at least one mediating compound which enhances the oxidation activity of the enzyme to be present in the reaction mixture. Said at least one mediating compound should desirably be present in the reaction mixture in an amount of from 0.001 to 10 mg per kg of 4-acetoxy-2-methylene butan-1-ol.

Commonly such a mediating compound will selectively bind to the compound to be oxidized. The degree of binding of the mediating compound (A) to the compound to be oxidized (B) may be quantified by the chemical equilibrium constant (K_{d}) resulting from the following binding reaction:

$$[ A ] + [ B ] [ A \text{::} B ]$$

The chemical equilibrium constant (K_{d}) is then given by:

$${K}_{d} = ( [ A ] \times [ B ] ) / [ A \text{::} B ]$$

It is preferred herein that the mediating compound will have a chemical equilibrium constant (K_{d}) for the compound to be oxidized of less that 1×10⁻⁴ and preferably less than 1×10⁻⁶.

Without intention to limit the present invention, suitable classes of mediating compounds include: oxidized nicotinamide cofactors; antibodies and fragments thereof which retain their binding properties; peptides; and, pepidomimics, wherein peptides are modified by the incorporation of non-natural amino acids and/or non-natural chemical linkages between the amino acids. Further, systems that include a laccase may comprise the known compounds of laccase mediator systems (LMS) of which mention may be made of: HBT (1-hydroxybenzotriazole); ABTS [2,2′-azinobis(3-ethylbenzothiazoline-6-sulfonic acid)]; NHA (N-hydroxyacetanilide); NEIAA (N-acetyl-N-phenylhydroxylamine); HBTO (3-hydroxy 1,2,3-benzotriazin-4(3H)-one); syringaldazine; and, VIO (violuric acid). And instructive disclosures of mediating compounds include: WO 94/04678 A (Casterman et al.); WO-94/25591 A (Unilever PLC et al.); and, WO 94/29457 A (Unilever PLC).

As noted above, the 4-acetoxy-2-methylene butan-1-ol (III) is contacted with the enzyme and mediator under aerobic conditions. The most economical source of oxygen is air which presents the advantages that air is easily obtained from the atmosphere at no cost, is non-toxic and need not be removed after the reaction. Alternatively, one may use molecular oxygen per se or employ a molecular oxygen liberating system such as a mixture of catalase enzyme and hydrogen peroxide.

In an illustrative practice, the introduced air or oxygen or the oxygen generated in situ is mixed into the reaction mixture under agitation for a period of time sufficient to allow for the conversion of substantially all of the 4-acetoxy-2-methylene butan-1-ol (III) into 4-acetoxy-2-methylene butyric acid (V). A typical reaction period will be less than 24 hours, for example from 1 to 5 hours.

The reaction mixture may further comprise a buffered solution to ensure that the pH thereof remains within a suitable range for the enzyme(s) to be active. Most laccases, for instance, exhibit pH optima in the acidic pH range while either demonstrating low activity or being inactive at a pH of 7. That said, the pH of the reaction mixture will commonly be retained in the range from 5 to 9, for example from 5.5 to 8.5. Without intention to limit the buffers used for such ranges, mention may be made of: imidazole; 1,4-piperazinediethanesulfonic acid (PIPES); 4-morpholinepropanesulfonic acid (MOPS); 4-(2-hydroxyethyl)-1-piperazineethane-sulfonic acid (HEPES); triethanolamine; tris(hydroxymethyl)aminomethane (TRIS); alkali metal phosphates; citric acid; and, trisodium citrate.

The reaction mixture is maintained at a temperature which allows for the reaction to proceed substantially to completion. The enzymes may be active within a temperature ranging of from 0 to 80° C. but it will be more common to employ a temperature in the range from 10 to 50° C. The use of a temperature in the range from 15 to 35° C. is advantageous as it negates the need to substantially heat or cool the reaction mixture from room temperature.

The progress of the reaction(s) of step c) can be monitored by known techniques of which mention may be made of ¹H NMR, Fourier Transform Infrared Spectroscopy, Ultra Performance Liquid Chromatography (UPLC), gas chromatography or thin layer chromatography (TLC).

The 4-acetoxy-2-methylene butyric acid (V) product of step c) may be isolated from the reactor. The isolated compound (V) may optionally be purified using methods known in the art including, but not limited to, solvent extraction, filtration, evaporation, distillation, crystallization and chromatography.

### Step c) Whole Cell Biotransformation

Two alternatives are envisaged for the performance of this step by whole cell biotransformation. In a first embodiment for the production of 4-acetoxy-2-methylene-butyric acid, the following sub-steps are carried out:
c) i) contacting a cell (CC) either with a culture medium containing 4-acetoxy-2-methylene-butan-1-ol (III) or with a culture medium contiguous with an organic phase containing 4-acetoxy-2-methylene-butan-1-ol under conditions that enable the cell to form 4-acetoxy-2-methylene-butyric acid (V) from 4-acetoxy-2-methylene-butan-1-ol (III); and optionally,
c) ii) isolation of the resultant 4-acetoxy-2-methylene-butyric acid (V),
wherein said cell (CC) exhibits activity of at least one enzyme exhibiting oxidizing activity.

As a preliminary point regarding this embodiment, it is not precluded that the aforementioned cell (CB) of step b) and the cell (CC) of step c) be the same: a singular cell may possess the relevant enzymatic activity of both said steps. That aside, the utilized cell (CC) of step c) i) may be a wild type cell or non-recombinant, laboratory cell which possesses a gene encoding at least one enzyme exhibiting oxidizing activity: in this regard, the cell may optionally possess an Alk operon, in particular an Alk operon containing the gene products AlkB, AlkH and AlkJ. Mention may again be made of the Pseudomonas putida in this context. In the alternative, the utilized cell (CC) of this embodiment may be genetically modified relative to its wild type so that, in comparison with said wild type, it is able to produce more 4-acetoxy-2-methylene butyric acid starting from 4-acetoxy-2-methylene butan-1-ol. This comparison is intended to encompass both: a) that case where the wild type of the genetically modified cell produces detectable amounts of 4-acetoxy-2-methylene butyric acid; and, B) that case where the wild type of the genetically modified cell is not able to form any detectable amount of 4-acetoxy-2-methylene butyric acid. Thus, as regards said second category of cells P), it is only following the genetic modification of the wild type to produce the cell used in step c) that a detectable amount of 4-acetoxy-2-methylene butyric acid is formed.

In a second embodiment of step c) for the production of 4-acetoxy-2-methylene-butyric acid, the following sub-steps are carried out:
c) i) contacting a cell (CC1) with a culture medium containing 4-acetoxy-2-methylene-butan-1-ol (III) or with a culture medium contiguous with an organic phase containing 4-acetoxy-2-methylene-butan-1-ol under conditions that enable the cell to form 4-acetoxy-2-methylene-butan-1-al (IV) from 4-acetoxy-2-methylene-butan-1-ol;
c) ii) optional isolation of the resultant 4-acetoxy-2-methylene-butan-1-al (IV);
c) iii) contacting a cell (CC2) with a culture medium containing said 4-acetoxy-2-methylene-butan-1-al (IV) or with a culture medium contiguous with an organic phase containing 4-acetoxy-2-methylene-butan-1-al (IV) under conditions that enable the cell to form 4-acetoxy-2-methylene butyric acid (V) from 4-acetoxy-2-methylene-butan-1-al; and,
c) iv) isolation of the resultant 4-acetoxy-2-methylene butyric acid (V),
wherein said cells (CC1, CC2) each exhibit increased activity of at least one enzyme exhibiting oxidizing activity.

As noted above, step c) ii) need not be performed. Where the cell (CC1) secretes 4-acetoxy-2-methylene-butan-1-al (IV) into the culture medium, cell (CC2) may be disposed within the same bioreactor and no intermediate isolation of 4-acetoxy-2-methylene-butan-1-al need be performed. Where either cell (CC2) is to be disposed in a distinct, downstream bioreactor or the host cell (CC1) producing 4-acetoxy-2-methylene-butan-1-al does not secrete it, step c) ii) may be performed to isolate that aldehyde.

As a preliminary point regarding this embodiment, it is not precluded that the aforementioned cell (CB) of step b) and the cell (CC1) of step c) i) be the same: a singular cell may possess the relevant enzymatic activity of both said steps. That aside, the utilized cell (CC1 of step c) i) may be a wild type cell or non-recombinant, laboratory cell which possesses a gene encoding at least one enzyme exhibiting oxidizing activity: in that regard, the cell (CC1) may optionally possess an Alk operon, in particular an Alk operon containing the gene products AlkH and AlkJ. Mention may again be made of the Pseudomonas putida in this context. In the alternative, the utilized cell (CC1) may be genetically modified relative to its wild type so that, in comparison with said wild type, it is able to produce more 4-acetoxy-2-methylene butan-1-al starting from 4-acetoxy-2-methylene butan-1-ol.

The utilized cell (CC2) may be a wild type cell or non-recombinant, laboratory cell which possesses a gene encoding at least one enzyme exhibiting oxidizing activity: in that regard, the cell (CC2) may optionally possess an Alk operon, in particular an Alk operon containing the gene products AlkH and AlkJ. In the alternative, the utilized cell (CC2) may be genetically modified relative to its wild type so that, in comparison with said wild type, it is able to produce more 4-acetoxy-2-methylene butyric acid starting from 4-acetoxy-2-methylene butan-1-al.

As stated above, any comparison between a recombinant and wild type cell is intended to encompass both: a) that case where the wild type of the genetically modified cell produces detectable amounts of the target product; and, p) that case where the wild type of the genetically modified cell is not able to form any detectable amount of the target product. Thus, as regards said second category of cells R), it is only following the genetic modification of the wild type to produce the cell used in steps c)i) and c)iii) that a detectable amount of the target product is formed.

It is preferred for each genetically modified cell (CC, CC1, CC2) to have been genetically modified so that, in a defined time interval of 24 hours, it forms at least 10 times, for example at least 100 times or at least 1000 times more of the target product than the wild-type cell. The increase in product formation may be determined by separately cultivating the cell(s) used according to step c) of the present disclosure and the wild-type cell under the same initial cell density, nutrient medium and culture conditions for the specified time interval and then determining the amount of the target product in each nutrient medium.

The cells (CC, CC1, CC2) used in step c) of the present disclosure may, independently, be prokaryotes or eukaryotes. They may be mammalian cells—including human cells—plant cells or microorganisms, such as fungi, molds or bacteria, wherein preferred microorganisms are those that have been deposited with Deutsche Sammlung von Mikroorganismen und Zellkulturen GmbH (DSMZ). Whilst yeasts may be important microorganisms (fungi) in this context, it is preferred to use bacteria and an instructive reference may therefore be made to http://www.dsmz.de/species/bacteria.htm.

Without intention to limit the present invention, cells (CC, CC1, CC2) that may be genetically modified and used in the step c) of the present disclosure are preferably independently selected from the genera: Corynebacterium; Brevibacterium; Bacillus; Lactobacillus; Lactococcus; Candida; Pichia; Kluveromyces; Saccharomyces; Escherichia; Zymomonas; Yarrowia; Methylobacterium; Ralstonia; Pseudomonas; Burkholderia; and, Clostridium. A particular preference may be mentioned for the use of cells (CC, CC1, CC2) independently selected from the group consisting of Escherichia coli, Corynebacterium glutamicum and Pseudomonas putida. Good results have been obtained with the E. coli BL21 strain.

In comparison with its wild type, a genetically modified cell (CC) according to step c) must exhibit increased activity of at least one of: an alcohol dehydrogenase (ADH) enzyme; an alcohol oxidase (AlcOx) enzyme; an aldehyde dehydrogenase (AlDH) enzyme; and, a laccase. In comparison with its wild type, the genetically modified cells (CC1) must exhibit increased activity of at least one of: an alcohol dehydrogenase (ADH) enzyme; an alcohol oxidase (AlcOx) enzyme; and, a laccase. And, in comparison with its wild type, the genetically modified cells (CC2) must exhibit increased activity of at least one of: an aldehyde dehydrogenase (AlDH) enzyme; and, a laccase.

Without intention to limit the present invention, the enzyme alcohol dehydrogenase may be encoded by a homologue of the AlkJ gene and the aldehyde dehyrogenase may be encoded by the AlkH gene from Pseudomonas putida GP01. The DNA sequence information for AlkJ alcohol dehydrogenase and the AlkH dehydrogenase can be taken from the Pseudomonas putida OCT Plasmid alk gene cluster identified as GenBank AJ 245436.1. And enzymes that are encoded by nucleic acids that have at least 40%, for example at least 75% and preferably at least 90% identity to the listed sequences are suitable for use in the method of the present invention.

To achieve an increased intracellular activity of the aforementioned enzymes in the genetically modified cells (CC, CC1, CC2), one or more of the following measures may be employed: an increase of the copy number of the gene sequence(s) that code for the enzyme; the use of a strong promoter for the gene; the use of a stronger ribosome binding site; the use of codon optimization; and, the employment of a gene or allele that codes for a corresponding enzyme with increased activity.

Where applicable, genetically modified cells (CC, CC1, CC2) are produced by transformation, transduction and/or conjugation with a vector that contains the desired gene, an allele of this gene or parts thereof and a vector that makes expression of the gene possible. Heterologous expression is achieved through the integration of the gene or the alleles thereof in the chromosome of the cell or an extra-chromosomally replicating vector. Without intention to limit the present disclosure, WO2009/077461 (Evonik Degussa GmbH) provides an instructive reference of cellular genetic transformation.

The utilized cells (CC, CC1, CC2) can be brought into contact with said culture medium in one or more bioreactors: as noted above, cells CC1 and CC2 may be disposed within the same or distinct bioreactors depending upon the secretion of the target product by cells CC1, the process compatibility of the independent strains CC1 and CC2 and/or the isolation of the intermediate aldehyde before the second oxidation step c) iii). Within the one or more bioreactors, the cells (CC, CC1, CC2) are cultivated, either continuously or discontinuously in a batch process, a fed-batch process or a repeated-fed-batch process as previously described.

Within the or each bioreactor defined in these embodiments of step c), the bio-production system is maintained within a suitable temperature range and within a controlled dissolved-oxygen concentration range for a sufficient time to obtain a desired conversion of the starting alcohol or aldehyde to the target product(s). A temperature range of from 20° C. to 50° C., for example from 20° C. to 40° C., may be mentioned as being suitable. Anaerobic conditions may be maintained in the bioreactors used in this step but it is more typical to maintain aerobic conditions therein via the addition of oxygen or an oxygen containing gas, such as air. The dissolved oxygen levels of a liquid culture comprising a nutrient media may be monitored to maintain or confirm a desired aerobic, microaerobic or anaerobic condition.

It will again be evident to the skilled artisan that the culture medium used in this step must be suitable for the requirements of the microorganism(s) used to produce the desired end product(s). To form the culture medium, an appropriate combination may be made of: at least one growth factor or a precursor thereof; at least one nitrogen source; at least one phosphorous source; and, at least one source of trace metal selected from the group consisting of manganese, boron, cobalt, copper, molybdenum, zinc, calcium, magnesium, iron, nickel and combinations thereof. Suitable growth factors include but are not limited to amino acids and vitamins, such as biotin, vitamin B12, derivatives of vitamin B12, thiamin and pantothenate. Nitrogen-sources include but are not limited to peptones, yeast extract, meat extract, malt extract, corn-steep liquor, soybean flour, ammonium sulphate, ammonium chloride, ammonium phosphate, ammonium carbonate and ammonium nitrate. Phosphorous sources include but are not limited to phosphoric acid, sodium dihydrogen phosphate, potassium dihydrogen phosphate, disodium hydrogen phosphate or dipotassium hydrogen phosphate. It will further be recognized that the medium may contain adjuvants such as: pH adjuster regulators including mineral acids and bases, for instance sodium hydroxide, potassium hydroxide, ammonia, ammonia water; antifoaming agents; and, antibiotics to maintain plasmid stability.

The medium may be a defined medium. It is also noted that step b) may be performed utilizing a minimal medium or a supplemented minimal medium. Further, the use of commercially prepared media is not precluded and particular mention may be made of: Luria Bertani (LB) broth; M9 Minimal Media; Sabouraud Dextrose (SD) broth; Yeast Medium (YM) broth, and Yeast Synthetic Minimal Media (Ymin).

In the aforementioned final sub-steps of each embodiment above—respectively c) ii) or c) iv)—the resultant 4-acetoxy-2-methylene butyric acid may be isolated from the bioreactor. Dependent on the cell used, the isolation of 4-acetoxy-2-methylene butyric acid may either be from the culture medium if the host cell secretes it into the medium or directly from the host cell producing it, if it is not so-secreted. The isolated 4-acetoxy-2-methylene butyric acid may optionally be purified using methods known in the art including, but not limited to, solvent extraction, filtration, evaporation, distillation, crystallization and chromatography.

### Step D)

This step of the process provides for the conversion of 4-acetoxy-2-methylene butyric acid to α-methylene-γ-butyrolactone. There is no particular intention to limit the method by which this step is performed but two preferred embodiments are worthy of mention.

In a first embodiment, this step d) may be performed in a one-pot synthesis wherein 4-acetoxy-2-methylene butyric (V) acid is subjected to acidic conditions at a temperature of from 30 to 300° C., for example from 30 to 150° C., under which conditions hydrolysis occurs to yield γ-hydroxy-α-methylenebutyric acid as an intermediate product. Under such acidic conditions, γ-hydroxy-α-methylenebutyric acid then cyclizes to yield α-methylene-γ-butyrolactone.

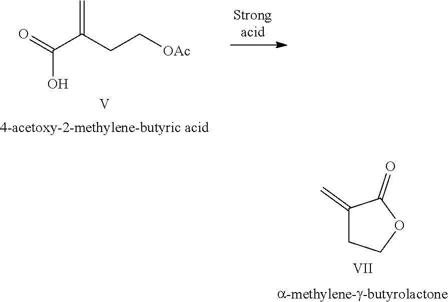

According to this embodiment of step d), either a Lewis acid or a Brönsted acid may be used and typically the molar ratio of said Lewis acid or said Brönsted acid to 4-acetoxy-2-methylene butyric acid should be in the range from 0.15-1:1. Suitable Lewis acids include but are not limited to: BF₃; AlCl₃; t-BuCl/Et₂AlCl; Cl₂/BCl₃; AlBr₃; AlBr₃·TiCl₄; ZrCl₄; I₂; SbCl₅; WCl₆; AlEt₂Cl; PF₅; VCl₄; AlEtCl₂; BF₃Et₂O; PCl₅; PCl₃; POCl₃; TiCl₆; FeCl₃; NiCl₂; and, SnCl₄. Suitable Brönsted acids may be either organic or inorganic acids but it is preferred to use Brönsted acids having a pKₐ value of at most 2.5, in particular those Brönsted acids having a pKₐ of from 2.5 to −10. Mention may be made of Brönsted acids selected from the group consisting of: sulfuric acid; phosphoric acid; hydrochloric acid, hydrobromic acid; hydroiodic acid; methanesulfonic acid; p-toluenesulfonic acid; perfluoroalkane sulfonic acids, such as trifluoromethane sulfonic acid (or triflic acid, CF₃SO₃H), C₂F₅SO₃H, C₄F₉SO₃H, C₅F₁₁SO₃H, C₆F₁₃SO₃H and C₈F₁₇SO₃H; tetrafluoroboric acid; trifluoroacetic acid; trichloroacetic acid; and, oxalic acid.

The progress of the above reaction can be monitored by known techniques of which mention may be made of ¹H NMR, Fourier Transform Infrared Spectroscopy, Ultra Performance Liquid Chromatography (UPLC), gas chromatography or thin layer chromatography (TLC). At an appropriate level of conversion, the reaction mixture may first be cooled to room temperature. The α-methylene-γ-butyrolactone is then subsequently isolated and may either be used in any subsequent synthesis step in crude form or further purified. Effective isolation and purification methods include, but not limited to, solvent extraction, filtration, evaporation, distillation, crystallization and chromatography.

In a second embodiment, the α-methylene-γ-butyrolactone is synthesized in a two-stage process via α-hydroxy-γ-methylenebutyric acid (VI). This two-stage process may be summarized by the following reaction scheme:

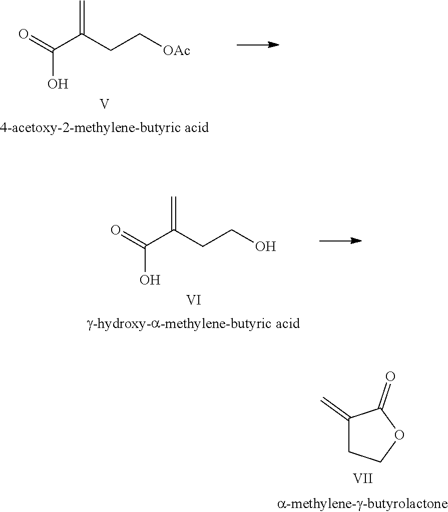

The first stage conversion of the acetoxy (OAc) groups to hydroxyl groups may be performed reductively, by basic hydrolysis, or enzymatically using an appropriate lipase or carboxyl esterase enzyme.

In a reductive conversion, 4-acetoxy-2-methylene butyric acid (V) is contacted with at least one hydride donor selected from borohydrides and aluminium hydride compounds. The hydride donor may for instance be selected from the group consisting of: potassium borohydride (KBH₄); sodium borohydride (NaBH₄); sodium triacetoxyborohydride (NaBH(OAc)₃); diborane (B₂H₆); sodium cyanoborohydride (NaBH₃CN); zinc borohydride (ZnBH₄); aluminium hydride (AlH₃); and, lithium aluminium hydride (LiAlH₄).

Preferably the hydride donor(s) is added to the reaction mixture in toto in an amount of at least 0.35 molar equivalents to the amount of 4-acetoxy-2-methylene butyric acid: for example, the hydride donor may be added in amount of from 0.5 to 2 molar equivalents to the amount of 4-acetoxy-2-methylene butyric acid. As would be recognized by the skilled artisan, the latter equivalence range for sodium borohydride (NaBH₄) would equate to a 2- to 8-fold excess of hydride (H) and serves to ensure complete reaction.

The selection of hydride donor(s) and/or the desired selectivity of the reaction are determinative of the solvent in which this reduction step should take place. Exemplary solvents for this stage of step d) are polar aprotic solvents including but not limited to: water; C₁-C₈ alkanols; acetonitrile; N,N-di(C₁-C₄)alkylacylamides, such as N,N-dimethylformamide (DMF) and N,N-dimethylacetamide (DMAc); hexamethylphosphoramide; N-methylpyrrolidone; pyridine; esters, such as (C₁-C₈)alkyl acetates, ethoxydiglycol acetate, dimethyl glutarate, dimethyl maleate, dipropyl oxalate, ethyl lactate, benzyl benzoate, butyloctyl benzoate and ethylhexyl benzoate; ketones, such as acetone, ethyl ketone, methyl ethyl ketone (2-butanone) and methyl isobutyl ketone; ethers, such as tetrahydrofuran (THF), 2-methyltetrahydrofuran (2-MeTHF) and 1,2-dimethoxyethane; 1,3-dioxolane; dimethylsulfoxide (DMSO); and, dichloromethane (DCM).

The reductive conversion of 4-acetoxy-2-methylene butyric acid (V) to γ-hydroxy-α-methylenebutyric acid (VI) does not require specialized equipment to exclude either water or air. Further, the reduction may be performed at a temperature of from 0 to 40° C., which range includes room temperature, which itself represents a preferred temperature condition.

In the conversion of 4-acetoxy-2-methylene butyric acid (V) to α-hydroxy-γ-methylenebutyric acid (VI) under basic conditions, a reaction mixture comprising water, 4-acetoxy-2-methylene butyric acid and a base is provided. The base should be present in the reaction mixture in an amount of from 1 to 5 molar equivalents to the amount of 4-acetoxy-2-methylene butyric acid. It is preferred that the base is present in an amount of from 1.5 to 3 molar equivalents or from 2 to 3 molar equivalents to the amount of 4-acetoxy-2-methylene butyric acid.

Without intention to limit the present invention, the base should desirably consist of at least one compound selected from alkali metal (C₁-C₄)alkoxides, alkali metal carbonates, alkaline earth metal carbonates or alkali metal hydroxides. A preference for the use of at least one of potassium carbonate, sodium carbonate and calcium carbonate is noted.

The basic hydrolysis may be performed under reflux conditions. As would be understood by the skilled artisan, the reflux temperature is dependent upon the reactants and the solvent present. That acknowledged it will be typical for the reflux temperature of this step—at atmospheric pressure—to be from 100 to 225° C., for example from 125 to 210° C. In an alternative methodology, the basic hydrolysis may be carried out in a sealed autoclave in which an autogenous pressure is generated in dependence upon the temperature at which the reaction is carried out. A temperature of from 100 to 250° C., for example from 100 to 225° C. may be established, such reaction temperatures generating autogenous pressures in the region of from 0.5 to 40 bar.

The progress of the above reaction can be monitored by known techniques of which mention may be made of ¹H NMR, Fourier Transform Infrared Spectroscopy, Ultra Performance Liquid Chromatography (UPLC) or thin layer chromatography (TLC). At an appropriate level conversion, the reaction mixture may first be cooled to room temperature. The α-hydroxy-γ-methylenebutyric acid (VI) is then subsequently isolated and may either be used in the subsequent synthesis step in crude form or further purified. Effective isolation and purification methods include, but not limited to, solvent extraction, filtration, evaporation, distillation, crystallization and chromatography.

The second stage of the aforementioned scheme can be performed enzymatically: WO2002101013A2 (E.I. Dupont de Nemours and Company), for example, discloses the formation of α-methylene-γ-butyrolactone from α-methylene-γ-hydroxybutyrate via a glycosylated intermediate under the action of UDP-glucose glucosyltransferase enzyme.

When the second stage of the aforementioned scheme is performed chemically, the α-hydroxy-γ-methylenebutyric acid (VI) is subjected to strongly acidic conditions at a temperature of from 30 to 300° C., for example from 30 to 150° C., under which conditions the hydroxy acid cyclizes to yield α-methylene-γ-butyrolactone (VII). In this stage, either a Lewis acid or a Brönsted acid may be used and typically the molar ratio of said Lewis acid or said Brönsted acid to 4-acetoxy-2-methylene butyric acid (V) should be in the range from 0.15-1:1. Suitable Lewis acids include but are not limited to: aluminum trichloride; tin tetrachloride; titanium tetrachloride; zirconium tetrachloride; iron trichloride; and, nickel dichloride. Suitable Brönsted acids may be either organic or inorganic acids but it is preferred to use Brönsted acids having a pKₐ value of at most 2.5, in particular those Brönsted acids having a pKₐ of from 2.5 to −10. Mention may be made of Brönsted acids selected from the group consisting of: sulfuric acid; phosphoric acid; hydrochloric acid, hydrobromic acid; hydroiodic acid; methanesulfonic acid; p-toluenesulfonic acid; tetrafluoroboric acid; trifluoroacetic acid; trichloroacetic acid; and, oxalic acid.

The progress of the second reaction can be monitored by the above referenced techniques. At an appropriate level conversion, the reaction mixture may first be cooled to room temperature. The α-methylene-γ-butyrolactone is then subsequently isolated and may either be used in any subsequent synthesis step in crude form or further purified. Effective isolation and purification methods include, but not limited to, solvent extraction, filtration, evaporation, distillation, crystallization and chromatography.

### Applications of α-Methylene-γ-Butyrolactone

### The Formation of Polymers Having Pendant Lactone Rings

The present invention disclosure provides for the polymerization of the above defined α-methylene-γ-butyrolactone (hereinafter MBL) to form polymers bearing pendant lactone rings. Broadly, the polymerization is performed under anionic conditions and the skilled artisan will select appropriate conditions so that the vinyl-addition pathway of polymerization predominates over the competing ring-opening polymerization pathway. The resultant polymer or copolymer (p-MBL) thus retains the lactone structure in its repeating unit.

The aforementioned monomer (MBL) may be incorporated into co-polymers (p-MBL) with at least one co-monomer. Most broadly, viable co-monomers are those that provide reasonable polymerization reaction rates under suitable, pragmatic anionic polymerization conditions. Desirably said at least one co-monomer is a non-carbonyl-providing, olefinically unsaturated monomer selected from the group consisting of: (meth)acrylonitrile; alkyl (meth)acrylate esters; (meth)acrylic acid; vinyl esters; and, vinyl monomers.

Suitable vinyl monomers include: ethylene; 1,3-butadiene; isoprene; styrene; divinyl benzene; heterocyclic vinyl compounds; and, vinyl halides such as chloroprene. Suitable vinyl esters include vinyl acetate, vinyl propionate, vinyl versatate and vinyl laurate. Suitable alkyl esters of acrylic acid and methacrylic acid are those derived from C₁ to C₁₄ alcohols and thereby include as non-limiting examples: methyl acrylate; methyl methacrylate; ethyl acrylate; ethyl methacrylate; n-butyl acrylate; n-butyl methacrylate; 2-ethylhexyl acrylate; 2-ethylhexyl methacrylate; isopropyl acrylate; hydroxyethyl methacrylate; hydroxypropyl methacrylate; isopropyl methacrylate; n-propyl acrylate; n-propyl methacrylate; and, di(meth)acrylate esters of alkane diols such as 1,6-hexane diol diacrylate.

The anionic polymerization of said α-methylene-γ-butyrolactone (MBL) and any co-monomers present is conducted in the presence of an initiator selected from the group consisting of: alkali metal organyls; alkali metal alkoxides; alkali metal thiolate; alkali metal amides; and, compounds of an element of group 3a of the Periodic Table of the Elements, preferably an aluminum or boron organyls and aluminum alkoxides, such as aluminum trimethoxide, aluminum triethoxide, aluminum tripropoxide, and, aluminum tributoxide.

There is no particular limitation on the amount of initiator used but it will be typically be from 0.0001 to 5 parts by weight, and preferably from 0.05 to 1 part by weight, based on 100 parts by weight of the monomers. Furthermore, the anionic polymerization may be performed in solution or in the melt without a solvent. When used, suitable solvents for the polymerization should be non-reactive, organic liquids capable of dissolving at least 1 wt. % and preferably over 10 wt. % polymers at 25° C. Dichloromethane and tetrahydrofuran (THF) may be mentioned as exemplary solvents.

The anionic polymerization process is desirably performed in the presence of a Lewis acid and, in particular, a “non-protic” Lewis acids which is not capable of functioning as a source of a proton (H+). Further, the anionic homo- or co-polymerization of α-methylene-γ-butyrolactone (MBL) should be performed under anhydrous conditions and in the absence of any compound having an active hydrogen atom, save for the deliberate inclusion of the initiating compound.

Whilst process pressure is not critical—in that the reaction may be performed at sub-atmospheric, atmospheric or super-atmospheric pressure—the anionic polymerization should typically be performed at a temperature of at least 25° C. Whilst the reaction temperature may be 200° C. or higher, it is preferred that the temperature does not exceed 200° C., 175° C. or even 150° C. in order inter alia: to maintain workable reactor pressures; to minimize the rate of polymer degradation and the concomitant formation of volatile impurities or other by-products; and, if applicable, to maintain adequate catalyst activity without deactivating or decomposing the catalyst.

The reaction product may be isolated and purified using methods known in the art. Whilst mention in this context may be made of extraction, evaporation, distillation and chromatography as suitable techniques, it is most convenient that the product of the reaction be isolated by distilling off the solvent and any unreacted starting materials under reduced pressure. Where it is intended that the (optionally purified) reaction product be stored upon production, the polymers should be disposed in a vessel with an airtight and moisture-tight seal.

Exemplary homo- or copolymers (p-MBL) derived by anionic polymerization of α-methylene-γ-butyrolactone obtained in accordance with the present invention may possess: i) a number-average molecular weight (Mn), as determined by gel permeation chromatography (GPC) in tetrahydrofuran using a polystyrene standard, of at least 2500 g/mol, for instance from 10000 to 150000 g/mol and preferably from 10000 to 100000 g/mol; ii) a glass transition temperature (Tg) of from 50 to 200° C., for example from 100 to 200° C.; and, iii) a polydispersity index (PDI) of from 1.1 to 2.0, for example from 1.10 to 1.90, and preferably from 1.10 to 1.80.

The polymers (p-MBL) of the present invention are considered to be versatile and thereby have a plethora of uses. For example, the lactone bearing polymers can be used to prepare ionic complexes with agents—including therapeutic agents such as a peptide—having a cationic moiety. The lactone ring(s) present in these polymers can also be opened by an alkali hydroxide to form an alkali metal salt of the corresponding hydroxycarboxylic acid. Furthermore, polymers containing lactone groups (p-MBL) can be crosslinked by means of functional compounds that can react with lactone. Unsubstituted primary or secondary amines and multifunctional amines—such hydroxyl substituted mono-, di- or tri-(C₁-C₁₂) alkylamines—are particularly desirable in this regard. And it is thus anticipated that the functionalized polymers (p-MBL) may find utility as a curable, crosslinkable or otherwise reactive component of a coating composition, a sealant composition or an adhesive composition.

Polyester Derivatives of α-Methylene-γ-Butyrolactone (MBL) or of the Homo- and Co-Polymers Thereof (p-MBL)

The present disclosure also provides for the formation of polyesters via the ring opening polymerization of either α-methylene-γ-butyrolactone (MBL) as obtained according to the defined process or of the pendant lactone groups of the aforementioned homo- and co-polymers of methylene-γ-butyrolactone (p-MBL) obtained by anionic polymerization. In this regard, said (co)polymer p-MBL may be considered as a reactant macromonomer.

The present disclosure further provides for the formation of block or random co-polyesters, whereby the lactone functional group of said methylene-γ-butyrolactone (MBL) or said (co-)polymer thereof (p-MBL) is used to regulate the ring opening polymerization of at least one further monomer (M2) selected from the group consisting of: cyclic carbonates; cyclic anhydrides; oxalates; and, cyclic esters having 5-, 6-, and/or 7-member rings. In particular, said at least one further monomer (M2) may be selected from the group consisting of: lactide; glycolide; ε-caprolactone; para-dioxanone; trimethylene carbonate; 1,4-dioxepan-2-one; 1,5-dioxepan-2-one; γ-butyrolactone; γ-methyl-α-methylene-γ-butyrolactone; α-bromo-γ-butyrolactone; α-hydroxy-γ-butyrolactone; α-acetyl-γ-butyrolactone; spirocyclic-γ-butyrolactone; γ-valerolactone; α-angelica lactone; and, β-angelica lactone.

Whilst there is no specific intention to limit the mechanism of ring opening polymerization employed in the present invention and whilst therefore ring opening polymerization of cyclic monomers by the anionic route, via basic catalysts is not strictly precluded, it is preferred herein for said polymerization to proceed by a cationic route, via acid catalysis. Broadly, any suitable acidic ring opening polymerization catalyst may be utilized herein and, equally, mixtures of catalysts may be employed. Both Lewis and Brönsted acids may be suitable in this context, but the latter are preferred as they tend to be effective at temperatures of less than 150° C. and are usually effective at temperatures of from 50 to 100° C.

Examples of suitable Lewis acids include but are not limited to: BF₃; AlCl₃; t-BuCl/Et₂AlCl; Cl₂/BCl₃; AlBr₃; AlBr₃·TiCl₄; I₂; SbCl₅; WCl₆; AlEt₂Cl; PF₅; VCl₄; AlEtCl₂; BF₃Et₂O; PCl₅; PCl₃; POCl₃; TiCl₆; and, SnCl₄. Examples of Brönsted acid or proton acid type catalysts—which may optionally be disposed on solid, inorganic supports—include, but are not limited to: HCl; HBr; HI; H₂SO₄; HClO₄; para-toluenesulfonic acid; trifluoroacetic acid; and, perfluoroalkane sulfonic acids, such as trifluoromethane sulfonic acid (or triflic acid, CF₃SO₃H), C₂F₅SO₃H, C₄F₉SO₃H, C₅F₁₁SO₃H, C₆F₁₃SO₃H and C₈F₁₇SO₃H. The most preferred of these strong acids is trifluoromethane sulfonic acid (triflic acid, CF₃SO₃H).

The catalysts for said ring opening polymerization may usually be employed at a concentration of from 1 to 1000 ppm by weight based on the total weight of the monomers to be polymerized. Preferably from 5 to 150 ppm by weight are used, most preferably from 5 to 50 ppm. The catalytic amount may be reduced when the temperature at which the monomers and the catalyst are contacted is increased.

The ring opening polymerization may conveniently be carried out at a temperature in the range from 10 to 150° C. Preferably, however, the temperature range is from 20 or 50 to 100° C. as obviating high temperatures can limit the loss of volatile monomers from the reaction mixture due to their lower boiling point.

The process pressure is not critical and as such, the polymerization reaction can be run at sub-atmospheric, atmospheric, or super-atmospheric pressures but pressures at or above atmospheric pressure are preferred. It is however important that the reaction should be performed under anhydrous conditions and in the absence of any compound having an active hydrogen atom.

The duration of the reaction is dependent on the time taken for the system to reach equilibrium. Equally, however, it is understood that the desired product can be obtained by stopping the equilibration at exactly the desired time: for example, the reaction can be monitored by analyzing viscosity over time or by analyzing monomer conversion using gas chromatography and the reaction stopped when the desired viscosity or monomer conversion is attained. These considerations aside, the polymerization reaction generally takes place for from 0.5 to 72 hours and more commonly from 1 to 30 or 1 to 20 hours. Acid catalysts present in the reaction mixture at the end of the polymerization reaction can easily be neutralized in order to stabilize the reaction product.

Upon completion of the polymerization, it is possible to remove any solid, suspended compounds by, for example, filtration, crossflow filtration or centrifugation. Further, the output of the polymerization may be worked up, using methods known in the art, to isolate and purify the hydroxyl-functionalized polyesters. Mention in this regard may be made of extraction, evaporation, distillation and chromatography as suitable techniques. Upon isolation, it has been found that typical yields of the hydroxyl-functionalized polyesters are at least 40% and often at least 60%.

Exemplary polyesters which may be derived by this ring opening polymerization process may possess a molecular weight (Mn) as determined as measured by gel permeation chromatography (GPC) in tetrahydrofuran using a polystyrene standard, of at least 5000, preferably from 10000 to 200000 g/mol. Moreover, the polymers may be characterized by a polydispersity index in the range from 1.0 to 2.5, preferably from 1.0 to 2.0.

### Further Polyester Formation

The methylene-γ-butyrolactone (MBL) or said (co-)polymer thereof (p-MBL) may have further utility as a monomer in an esterification, wherein the resultant copolymer comprises non-lactoyl units derived from at least two co-monomers which are capable of forming an ester bond. More particularly, those co-monomers comprise: i) at least one diol; and, (ii) at least one dicarboxylic acid or its ester forming derivative.

Suitable diols (i) for use in this context include saturated and unsaturated aliphatic and cycloaliphatic dihydroxy compounds as well as aromatic dihydroxy compounds. These diols preferably have a molecular weight of 250 daltons or less. When used herein, the term “diol” should be construed to include equivalent ester forming derivatives thereof, provided, however, that the molecular weight requirement pertains to the diol only and not to its derivative. Exemplary ester forming derivatives include the acetates of the diols as well as, for example, ethylene oxide or ethylene carbonate for ethylene glycol.

Preferred diols are those having from 2 to 10 carbon atoms. As examples of these diols there might be mentioned: ethylene glycol; propylene glycol; 1,3-propane diol; 1,2-butane diol; 2-methyl propanediol; 1,3-butane diol; 1,4-butane diol; 2,3-butanediol; neopentyl glycol; hexanediol; decanediol; hexamethylene glycol; cyclohexane dimethanol; resorcinol; and, hydroquinone. Mixtures of such diols may be employed, but in this regard, it is generally preferred that at least about 60 mol. % and preferably at least 80 mol. %, based on the total diol content, be the same diol.

Dicarboxylic acids (ii) which are suitable for use in the above context include aliphatic, cycloaliphatic, and/or aromatic dicarboxylic acids. These acids should preferably have molecular weight of less than 300 daltons. The term “dicarboxylic acids” as used herein includes equivalents of dicarboxylic acids having two functional carboxyl groups which perform substantially like dicarboxylic acids in reaction with glycols and diols in forming polyesters. These equivalents include esters and ester forming reactive derivatives, such as acid halides and anhydrides, provided however that the molecular weight preference mentioned above pertains to the acid and not to its equivalent ester or ester-forming derivatives. Thus, an ester of a dicarboxylic acid having a molecular weight greater than 300 daltons or an acid equivalent of a dicarboxylic acid having a molecular weight greater than 300 daltons are included provided the acid has a molecular weight below 300 daltons. Additionally, the dicarboxylic acids may contain any substituent groups(s) or combinations which do not substantially interfere with the polymer formation and use of the polymer of this invention.

Preferred dicarboxylic acids are those selected from the group comprising alkyl dicarboxylic acids having a total of 2 to 16 carbons atoms and aryl dicarboxylic acids having a total of from 8 to 16 carbon atoms. Representative alkyl dicarboxylic acids include: glutaric acid; adipic acid; pimelic acid; succinic acid; sebacic acid; azelaic acid; and, malonic acid. Representative aryl dicarboxylic acids include: terephthalic acid; phthalic acid; isophthalic acid; the dimethyl derivatives of said acids; and, mixtures thereof.

### Further Embodiment of the Disclosure

The present invention also provides a process for preparing a compound of Formula (BIII) from a compound of Formula (BII) by whole cell biotransformation:

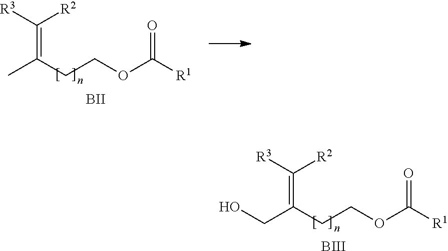

wherein:
n is an integer of from 0 to 8;
R¹ is C₁-C₄ alkyl;
R² is H or C₁-C₄ alkyl; and,
R³ is H or C₁-C₄ alkyl,
   said process comprising:
iii) contacting a cell (CB) with a culture medium containing said compound of Formula (BII) or with a culture medium contiguous with an organic phase containing said compound of Formula (BII) under conditions that enable the cell to form said compound of Formula (BIII) from said compound of Formula (BII); and optionally
iv) isolating the resultant compound of Formula (BIII), wherein said cell (CB) exhibits activity of at least one alkane monooxygenase enzyme which catalyzes the Cₙ₊₃-hydroxylation of said compound of Formula (BII).

The utilized cell (CB) of this step possesses the gene of an alkane monooxygenase, optionally as part of an Alk operon. In an embodiment, the utilized cell (CB) may be a wild type cell or non-recombinant, laboratory cell which possesses the gene of an alkane monooxygenase, optionally as part of an Alk operon, in particular an Alk operon containing the gene products AlkB, AlkH and AlkJ. Mention may again be made of the Pseudomonas putida, the wild type genotype of which contains two Alk operons: the first operon encodes the gene products AlkB, AlkF, AlkG, AlkH, AlkJ, AlkK and AlkL; the second operon encodes AlkS and AlkT, wherein AlkS has a regulatory function on the expression of the first alk operon.

In the alternative, the utilized cell (CB) may have been genetically modified relative to its wild type so that, in comparison with said wild type, it is able to produce more of the compound of Formula (BIII) starting from the compound of Formula (BII). This comparison is intended to encompass both: i) that case where the wild type of the genetically modified cell produces detectable amounts of the compound of Formula (BIII); and, ii) that case where the wild type of the genetically modified cell is not able to form any detectable amount of the compound of Formula (BIII). Thus, as regards said second category of cells ii), it is only following the genetic modification of the wild type to produce the cell used in this process step that a detectable amount of the compound of Formula (BIII) is formed.

It is preferred for the utilized cell (CB) to have been genetically modified so that, in a defined time interval of 24 hours, it forms at least 10 times, for example at least 100 times or at least 1000 times, more of the compound of Formula (BIII) than the wild-type cell. The increase in product formation may be determined by separately cultivating the cell used according to step b) of the present disclosure and the wild-type cell under the same initial cell density, nutrient medium and culture conditions for the specified time interval and then determining the amount of the target product in each nutrient medium.

The cells (CB) used in this step of the present disclosure may be prokaryotes or eukaryotes. They may be mammalian cells—including human cells—plant cells or microorganisms, such as fungi, molds or bacteria, wherein preferred microorganisms are those that have been deposited with Deutsche Sammlung von Mikroorganismen und Zellkulturen GmbH (DSMZ). Whilst yeasts may be important microorganisms (fungi) in this context, it is preferred to use bacteria and an instructive reference may therefore be made to http://www.dsmz.de/species/bacteria.htm.

The potential use of wild type Pseudomonas putida in the present invention has been noted above. Without intention to limit the present invention, preferred cells (CB) that may be genetically modified and used in this embodiment of the present disclosure may be selected from the genera: Corynebacterium; Brevibacterium; Bacillus; Lactobacillus; Lactococcus; Candida; Pichia; Kluveromyces; Saccharomyces; Escherichia; Zymomonas; Yarrowia; Methylobacterium; Ralstonia; Pseudomonas; Burkholderia; and, Clostridium. A particular preference may be mentioned for the use of cells (CB) selected from the group consisting of Escherichia coli, Corynebacterium glutamicum and Pseudomonas putida. Good results have been obtained with the E. coli BL21 strain.

In comparison with its wild type, a genetically modified cell (CB) according to this embodiment must exhibit increased activity of at least one of alkane monooxygenase enzyme which catalyzes the Cₙ₊₃-hydroxylation of the compound of Formula (BII): the direct Cₙ₊₃-hydroxylation of the compound of Formula (BII) leads to the compound of Formula (BIII).

The enzyme alkane monooxygenase may be encoded by the AlkB gene from Pseudomonas putida GP01, mutants and homologues thereof. For example, the enzyme alkane monooxygenase may be encoded by the AlkB gene from Pseudomonas putida GP01 selected from the group consisting of: the AlkBGT gene cluster; the AlkBGTJH gene cluster; and, the AlkBGTJHL gene cluster. The isolation of the respective gene sequence is described for example in van Beilen et al., “Functional Analysis of Alkane Hydroxylases from Gram-Negative and Gram-Positive Bacteria”, Journal of Bacteriology, Vol. 184 (6), pages 1733-1742 (2002). Moreover, the DNA sequence information encoding AlkB, AlkF, AlkG and AlkT were obtained from the Pseudomonas putida OCT Plasmid alk gene cluster identified as GenBank AJ-245436.1; and, the plasmid sequence for the pCom10 vector into which AlkBGT is cloned can be found in GenBank 302087.1.

Enzymes that are encoded by nucleic acid sequences that have at least 40%, preferably at least 50% and more preferably at least 75% identity to the identified sequences are suitable for use in the method of the present invention. For completeness, further exemplary homologues of the AlkB gene include but are not limited to: AlkB-P1 obtained from Pseudomonas putida P1; Alk1-MO obtained from Marinobacter hydrocarbonoclasticus; and, AlkB1 obtained from Alcanivorax borkumensis.

To achieve an increased intracellular activity of the aforementioned enzymes in the genetically modified cells (CB), one or more of the following measures may be employed: an increase of the copy number of the gene sequence(s) that code for the enzyme; the use of a strong promoter for the gene; the use of a stronger ribosome binding site; the use of codon optimization; the employment of a gene or allele that codes for a corresponding enzyme with increased activity; and, the employment of an altered amino acid sequence of the above mentioned enzymes, exhibiting increased activity, as described, for instance, in Koch et al., “In Vivo Evolution of Butane Oxidation by Terminal Alkane Hydroxylases AlkB and CYP153A6”, Applied and Environmental Microbiology, Vol 75 (2), pages 337-344 (2009).

Genetically modified cells used in the method according to the invention are produced by transformation, transduction and/or conjugation with an expression vector that contains the desired gene, an allele of this gene or parts thereof and a vector that makes expression of the gene possible. Heterologous expression is achieved through the integration of the gene or the alleles thereof in the chromosome of the cell or an extra-chromosomally replicating vector. Without intention to limit the present disclosure, WO2009/077461 (Evonik Degussa GmbH) provides an instructive reference of cellular genetic transformation.

The cells (CB) can be brought into contact with said culture medium in a bioreactor, and therefore cultivated, either continuously or discontinuously in a batch process, a fed-batch process or a repeated-fed-batch process, as described hereinabove in relation to step b).

Within the bioreactor, the bio-production system is maintained within a suitable temperature range and within a controlled dissolved-oxygen concentration range for a sufficient time to obtain the desired conversion (Cₙ₊₃-hydroxylation) of the compound (BII) substrate molecules to yield the chemical product (BIII). A temperature range of from 20° C. to 50° C., for example from 20° C. to 40° C., may be mentioned as being suitable for this step. Anaerobic conditions may be maintained in the bioreactors used in this step but it is more typical to maintain aerobic conditions therein via the addition of oxygen or an oxygen containing gas, such as air. The dissolved oxygen levels of a liquid culture comprising a nutrient media may be monitored to maintain or confirm a desired aerobic, microaerobic or anaerobic condition.

It will be evident to the skilled artisan that the culture medium used in this step must be suitable for the requirements of the microorganism(s) used to produce the desired end product (BIII). To form the culture medium, an appropriate combination may be made of: at least one growth factor or a precursor thereof; at least one nitrogen source; at least one phosphorous source; and, at least one source of trace metal selected from the group consisting of manganese, boron, cobalt, copper, molybdenum, zinc, calcium, magnesium, iron, nickel and combinations thereof. Suitable growth factors include but are not limited to amino acids and vitamins, such as biotin, vitamin B12, derivatives of vitamin B12, thiamin and pantothenate. Nitrogen-sources include but are not limited to peptones, yeast extract, meat extract, malt extract, corn-steep liquor, soybean flour, ammonium sulphate, ammonium chloride, ammonium phosphate, ammonium carbonate and ammonium nitrate. Phosphorous sources include but are not limited to phosphoric acid, sodium dihydrogen phosphate, potassium dihydrogen phosphate, disodium hydrogen phosphate or dipotassium hydrogen phosphate. It will further be recognized that the medium may contain adjuvants such as: pH adjuster regulators including mineral acids and bases, for instance sodium hydroxide, potassium hydroxide, ammonia, ammonia water; antifoaming agents; and, antibiotics to maintain plasmid stability.

The medium may be a defined medium. It is also noted that this embodiment may be performed utilizing a minimal medium or a supplemented minimal medium. Further, the use of commercially prepared media is not precluded and particular mention may be made of: Luria Bertani (LB) broth; M9 Minimal Media; Sabouraud Dextrose (SD) broth; Yeast Medium (YM) broth, and Yeast Synthetic Minimal Media (Ymin).

The compound (BIII) may optionally be isolated from the bioreactor, which isolation may either be from the culture medium if the host cell secretes it into the medium or directly from the host cell producing it, if it is not so-secreted. The isolated compound (BIII) may subsequently be purified using methods known in the art including, but not limited to, solvent extraction, filtration, evaporation, distillation, crystallization and chromatography.

The following examples are illustrative of the present invention and are not intended to limit the scope of the invention in any way.

### EXAMPLES

Unless stated otherwise, all chemicals and compounds described hereinafter were purchased from Merck/Sigma-Aldrich, TCI chemicals or Carl Roth at the highest purity commercially available.

NMR Spectroscopy: ¹H- and ¹³C-spectra were recorded using an Avance™ III 300 MHz FT NMR spectrometer; for the analysis, from 5 to 10 mg of analyte was dissolved in CDCl₃ and the obtained spectroscopic data was compared to literature.

Gas Chromatoaraphy-Mass Spectrometry (GC-MS): This analysis was used to qualitatively confirm the formation of the desired products. Conditions for analytics were established using pure compounds. All measurements were performed on a Shimadzu GCMS-QP2010 SE instrument equipped with an AOC-20i/s autosampler and injector unit together with a Zebron ZB-5MSi capillary column (30 m×0.25 mm×0.25 μm, Phenomenex). The test parameters are detailed in Table 1 hereinbelow:

TABLE 1
Parameters of GC-MS Analysis

| GC Parameters | | |
|---|---|---|
| Flow Control Mode | Linear velocity (39.5 cm sec⁻¹), Carrier gas: He | |
| Total flow | 15 | mL min⁻¹ |
| Column flow | 1.21 | mL min⁻¹ |
| Injection temperature | 250° | C. |
| Injection volume | 1 | μL |
| Split ratio | 9.1 | |
| Temperature program | 5 min at 50° C., 40° C./min to | |
| | 300° C., 5 min at 300° C. | |
| MS Parameters | | |
| Ion source temperature | 250° | C. |
| Interface temperature | | |

| | |
|---|---|
| Mode | Scan, 30-300 m z⁻¹ |

Total program time: 16.25 min

Gas Chromatography-Flame Ionization Detector (GC-FID): This analysis was used to quantitatively confirm the formation of the desired products. Conditions for analytics were established using pure compounds. All measurements were performed on a Shimadzu Nexis GC-2030 equipped with an AOC-20i Plus autosampler and injector unit and a Zebron ZB-5MSi capillary column (30 m×0.25 mm×25 μm, Phenomenex). The concentrations of the analytes were calculated from calibration curves generated by measuring samples with known concentrations of the pure compounds (0-6 mM; normalization with internal standard, ISTD) extracted from resting cell buffer. Calibration variables of compounds that were not available were based on structurally similar chemicals; in particular, the calibration curve for isoprenyl acetate was applied for 4-acetoxy-2-methylene butan-1-ol.

The test parameters are detailed in Table 2 hereinbelow:

TABLE 2
Parameters of GC-FID Analysis

| GC Parameters | | |
|---|---|---|
| Flow Control Mode | Linear velocity (22 cm sec⁻¹), Carrier gas: N₂ | |
| Total flow | 15.9 | mL min⁻¹ |
| Column flow | 1.18 | mL min⁻¹ |
| Injection temperature | 250° | C. |
| Injection volume | 1 | μL |
| Split ratio | 10.0 | |
| Temperature program | 1 min at 50° C., 20° C./min to | |
| | 250° C., 2 min at 250° C. | |
| FID Parameters | | |
| FID Temperature | 320° | C. |

### Example 1: Synthesis of Isoprenyl Acetate (II)

To a round-bottom flask of 50 mL equipped with magnetic stirring, 60.9 mmol of acetic anhydride (Ac₂O) and 0.58 mmol of Mg(ClO)₄ were added. The reaction was initiated by the dropwise addition of isoprenol (I, 58 mmol). The reaction was monitored by Thin Layer Chromatography (cyclohexane:ethyl acetate (EtOAc) of 3:1).

After 30 minutes the reaction was complete, aqueous NaHCO₃ was added and the product was extracted with diethyl ether (Et₂O). The organic layer was dried over MgSO₄ and the solvent was evaporated to give the pure ester (Yield: 88%). The formation of isoprenyl acetate (II) was verified by GC-MS and NMR-spectroscopy against known literature [5].

### Example 2

### 2a) Cloning and Expression of AlkBFGT

AlkBFGT was cloned analogously as described by Schrewe et al. [1] and van Nuland et al. [2] into the broad-host vector pCom10_AlkL [3] by excising AlkL and giving the vector pBT10.

Synthetic DNA-fragments encoding alkBFG and alkT were obtained from Integrated DNA Technologies. The fragments were sub-cloned into the pJET1.2 vector using the Thermo Scientific CloneJET PCR Cloning Kit. The alkBFG and alkTfragments were inserted into the pCom10 vector backbone via the FastCloning method [4]. Correct assembly of pBT10 was verified by colony PCR as well as by Sanger Sequencing.

E. coli BL21(DE3) harboring the pBT10 plasmid was used to express AlkBFGT at similar condition as described in literature ([1], [2]). E. coli BL21(DE3) pCom10_AlkL was used for empty vector control experiments. Strains were grown in either Luna-Bertani (LB) or M9 Minimal Medium (1×M9 salts, 0.5% glucose, 2 mM MgSO₄, 1 ml L⁻¹ of US^{Fe} trace elements) supplemented with 50 μg mL⁻¹ kanaymycin for selection. 5 mL of LB medium were inoculated with a single colony and incubated overnight at 30° C. and constant agitation (120 rpm). 500 μL of the overnight culture (ONC) were transferred to 50 mL of M9 Minimal Medium in a 100 mL shake flask and the culture was again incubated overnight at 30° C.

For the expression, from 100 to 400 mL of M9 Minimal Medium were inoculated with the M9-preculture to an optical density, as measured at a wavelength of 600 nm (OD₆₀₀), of 0.15 and grown at 30° C. until an OD₆₀₀ of from 0.4 to 0.5 was achieved. By addition of 0.05% (v/v) dicyclopropyl ketone (DCPK), recombinant gene expression was induced. After 4 hours of expression at 30° C., the cells were harvested by centrifugation (4400×g, 4° C., 15 min). 2b) Whole-cell biotransformation and isolation of 4-acetoxy-2-methylene-butan-1-ol

After expression and harvest, the cells were re-suspended in the resting cell buffer (50 mM KPi, pH 7.4, 1% glucose, 2 mM MgSO₄) to an OD₆₀₀ of 10. The cells were adapted to the reaction conditions for 5 to 10 minutes. The reaction described below was then performed in biological triplicate.

The reaction was performed in a 1.5 mL glass vial in a total volume of 300 μL. The reaction was started by the addition of 5 mM of the substrate isoprenyl acetate (II), stock 200 mM in EtOH, 2.5% (v/v) EtOH in the reaction). The cells were incubated for 24 hours at 25° C. under constant agitation (180 rpm). To stop the reaction, the mixture was stored at −20° C. until further use. Substrate conversion was analyzed by GC-MS and GC-FID.

As a negative control, E. coli pCom10_AlkL were used instead of E. coli pBT10. To confirm general activity of the AlkBFGT system, conversion of n-octane to 1-octanol and further overoxidation to octanal and octanoic acid by AlkBFGT was performed as model reaction.

To verify the formation of 4-acetoxy-2-methylene-butan-1-ol (III) by NMR spectroscopy, the reaction was scaled up to a total volume of 20 mL and was performed in 100 mL shake flask conducted for 24 hours of reaction time. The reaction mixture was stored at −20° C. until further use.

The product was extracted with ethyl acetate (EtOAc) from the cell suspension. The organic layer was dried over MgSO₄ and the solvent was evaporated. The crude extract was subjected to column chromatography (stationary phase: silica 60; solvent:cyclohexane/EtOAc 4:1) to yield the purified 4-acetoxy-2-methylene-butan-1-ol (III). The solvent of fractions containing compound III was evaporated to give pure III, the yield of which is reported in Table 3 below. The fractions were analyzed by TLC (cyclohexane:EtOAc, 2:1; detection by KMnO₄ staining). Spectroscopic data was compared to spectroscopic data in literature ([6], [7]).

For the analysis of the whole-cell biotransformation, 250 μL of reaction mixture were acidified with 25 μL of 2 M HCl and extracted with 250 μL EtOAc containing 1 mM of methylbenzoate as internal standard by vigorous shaking. Phase separation was achieved by centrifugation (16,000×g, 4° C., 7 min).

The organic phase was dried over MgSO₄. 200 μL of extract were directly subjected to GC-MS or GC-FID analysis for qualitative or quantitative analysis, respectively.

The yield data reported in Table 3 herein below is represented as the arithmetic mean of the triplicate reactions together with the standard deviation (SD).

TABLE 3

| | | | | | Standard |
|---|---|---|---|---|---|
| | | Gene | AlkL Co- | Yield | Deviation |
| Identifier | Insert | Origin | expression | (mM)¹ | (mM) |
| pBT10 | PpGPo1AlkB | P. Putida | No | 0.55 | 0.12 |
| | | GPo1 | | | |

¹Yields reported based on 5 mM of the starting substrate, isoprenyl acetate (II)

### Example 3

3a) Cloning and Expression of AlkBFGT with Co-Expression of AlkL

AlkBFGTL was cloned analogously as described by Schrewe et al. [1] and van Nuland et al. [2] into the broad-host vector pCom10_AlkL [3] giving the vector pBTL10.

Synthetic DNA-fragments encoding alkBFG and alkT were obtained from Integrated DNA Technologies. The gene encoding AlkL was amplified from pCom10_AlkL. The fragments were sub-cloned into the pJET1.2 vector using the Thermo Scientific CloneJET PCR Cloning Kit. The alkBFG, alkT and alkL fragments were inserted into the pCom10 vector backbone via the FastCloning method [4]. Correct assembly of pBTL10 was verified by colony PCR as well as by Sanger Sequencing.

E. coli BL21(DE3) harboring the pBTL10 plasmid was used to express AlkBFGTL at similar conditions as described in literature ([1], [2]). E. coli BL21(DE3) pCom10_AlkL was used as the empty vector control. Strains were grown in either Luna-Bertani (LB) or M9 Minimal Medium (1×M9 salts, 0.5% glucose, 2 mM MgSO₄, 1 ml L⁻¹ of US^{Fe} trace elements) supplemented with 50 μg mL⁻¹ kanaymycin for selection. 5 mL of LB medium were inoculated with a single colony and incubated overnight at 30° C. and constant agitation (120 rpm). 500 μL of the overnight culture (ONC) were transferred to 50 mL of M9 Minimal Medium in a 100 mL shake flask and the culture was again incubated overnight at 30° C.

For the expression, from 100 to 400 mL of M9 Minimal Medium were inoculated with the M9-preculture to an optical density, as measured at a wavelength of 600 nm (OD₆₀₀), of 0.15 and grown at 30° C. until an OD₆₀₀ of from 0.4 to 0.5 was achieved. By addition of 0.05% (v/v) dicyclopropyl ketone (DCPK), recombinant gene expression was induced. After 4 hours of expression at 30° C., the cells were harvested by centrifugation (4400×g, 4° C., 15 min).

3b) Whole-Cell Biotransformation and Isolation of 4-acetoxy-2-methylene-butan-1-ol

After expression and harvest, the cells were re-suspended in the resting cell buffer (50 mM KPi, pH 7.4, 1% glucose, 2 mM MgSO₄) to an OD₆₀₀ of 10. The cells were adapted to the reaction conditions for 5 to 10 minutes. The reaction described below was then performed in biological triplicate.

The reaction was performed in a 1.5 mL glass vial in a total volume of 300 μL. The reaction was started by the addition of 5 mM of the substrate (isoprenyl acetate (II), stock 200 mM in EtOH, 2.5% (v/v) EtOH in the reaction). The cells were incubated for 24 hours at 25° C. and under constant agitation (180 rpm). To stop the reaction, the mixture was stored at −20° C. until further use. Substrate conversion was analyzed by GC-MS and GC-FID.

As a negative control, E. coli pCom10_AlkL were used instead of E. coli pBTL10. To confirm general activity of the AlkBFGTL system, conversion of n-octane to 1-octanol and further overoxidation to octanal and octanoic acid by AlkBFGTL was performed as model reaction.

To verify the formation of 4-acetoxy-2-methylene-butan-1-ol (III) by NMR spectroscopy, the reaction was scaled up to a total volume of 20 mL and was performed in 100 mL shake flask conducted for 24 hours of reaction time. The reaction mixture was stored at −20° C. until further use.

The product was extracted with ethyl acetate (EtOAc) from the cell suspension. The organic layer was dried over MgSO₄ and the solvent was evaporated. The crude extract was subjected to column chromatography (stationary phase: silica 60; solvent:cyclohexane/EtOAc 4:1) to yield the purified 4-acetoxy-2-methylene-butan-1-ol (III). The solvent of fractions containing compound III was evaporated to give pure III, the yield of which is reported in Table 4 below. The fractions were analyzed by TLC (cyclohexane:EtOAc, 2:1; detection by KMnO₄ staining). Spectroscopic data was compared to spectroscopic data in literature ([6], [7]).

For the analysis of the whole-cell biotransformation, 250 μL of reaction mixture were acidified with 25 μL of 2 M HCl and extracted with 250 μL EtOAc containing 1 mM of methylbenzoate as internal standard by vigorous shaking. Phase separation was achieved by centrifugation (16,000×g, 4° C., 7 min). The organic phase was dried over MgSO₄. 200 μL of extract were directly subjected to GC-MS or GC-FID analysis for qualitative or quantitative analysis, respectively.

The yield data reported in Table 4 herein below is represented as the arithmetic mean of the triplicate reactions together with the standard deviation (SD).

TABLE 4

| | | | | | Standard |
|---|---|---|---|---|---|
| | | Gene | AlkL Co- | Yield | Deviation |
| Identifier | Insert | Origin | expression | (mM)¹ | (mM) |
| pBTL10 | PpGPo1AlkB | P. Putida | Yes | 0.97 | 0.04 |
| | | GPo1 | | | |

¹Yields reported based on 5 mM of the starting substrate, isoprenyl acetate (II)

### Example 4

### 4a) Cloning and Expression of AlkB Mutants

Two AlkBFGT DNA fragments, each possessing an AlkB mutant, were cloned as described by Schrewe et al. [1] and van Nuland et al. [2] into the broad-host vector pCom10_AlkL [3] by excising AlkL and giving the vector pBT10_AlkBmutant. The following AlkB mutants are identified as follows: AlkB(I233V), mutant disclosed in Koch et al. [8]; and, AlkB(F164L), a single mutant identified from docking studies using isoprenyl acetate as ligand in which the amino acid phenylalanine (Phe) of the wild-type AlkB is exchanged to leucine (Leu).

Synthetic DNA-fragments encoding alkBFG and alkT were obtained from Integrated DNA Technologies. Where applicable, the gene encoding AlkL was amplified from pCom10_AlkL. The fragments were sub-cloned into the pJET1.2 vector using the Thermo Scientific CloneJET PCR Cloning Kit. The alkBFG, alkT and, where applicable, the AlkL fragments were inserted into the pCom10 vector backbone via the FastCloning method [4] resulting the pBT10 vector. The mutations were introduced by site-directed mutagenesis. Correct assembly and successful mutagenesis of pBT10_AlkBmutant was verified by colony PCR as well as by Sanger Sequencing.

E. coli BL21(DE3) harboring the pBT10_AlkBmutant plasmid was used to express AlkB(mut)FGT or, where applicable, AlkB(mut)FGTL at similar conditions as described in literature ([1], [2]). E. coli BL21(DE3) pCom10_AlkL was used as the empty vector control. Strains were grown in either Luna-Bertani (LB) or M9 Minimal Medium (1×M9 salts, 0.5% glucose, 2 mM MgSO₄, 1 ml L⁻¹ of US^{Fe} trace elements) supplemented with 50 μg mL⁻¹ kanaymycin for selection. 5 mL of LB medium were inoculated with a single colony and incubated overnight at 30° C. and constant agitation (120 rpm). 500 μL of the overnight culture (ONC) were transferred to 50 mL of M9 Minimal Medium in a 100 mL shake flask and the culture was again incubated overnight at 30° C.

For the expression, from 100 to 400 mL of M9 Minimal Medium were inoculated with the M9-preculture to an optical density, as measured at a wavelength of 600 nm (OD₆₀₀), of 0.15 and grown at 30° C. until an OD₆₀₀ of from 0.4 to 0.5 was achieved. By addition of 0.05% (v/v) dicyclopropyl ketone (DCPK), recombinant gene expression was induced. After 4 hours of expression at 30° C., the cells were harvested by centrifugation (4400×g, 4° C., 15 min). 4b) Whole-cell biotransformation and isolation of 4-acetoxy-2-methylene-butan-1-ol

After expression and harvest, the cells were re-suspended in the resting cell buffer (50 mM KPi, pH 7.4, 1% glucose, 2 mM MgSO₄) to an OD₆₀₀ of 10. The cells were adapted to the reaction conditions for 5 to 10 minutes. The reaction described below was then performed in biological triplicate.

The reaction was performed in a 1.5 mL glass vial in a total volume of 300 μL mL. The reaction was started by the addition of 5 mM of the substrate isoprenyl acetate (II), stock 200 mM in EtOH, 2.5% (v/v) EtOH in the reaction). The cells were incubated for 24 hours at 25° C. under constant agitation (180 rpm). To stop the reaction, the mixture was stored at −20° C. until further use. Substrate conversion was analyzed by GC-MS and GC-FID.

As a negative control, E. coli pCom10_AlkL were used instead of E. coli pBT10_AlkBmutant. To confirm general activity of the AlkBFGT or, where applicable, AlkBFGTL system, conversion of n-octane to 1-octanol and further overoxidation to octanal and octanoic acid by AlkBFGT(L) was performed as model reaction.

To verify the formation of 4-acetoxy-2-methylene-butan-1-ol (III) by NMR spectroscopy, the reaction was scaled up to a total volume of 20 mL and was performed in 100 mL shake conducted for 24 hours of reaction time. The reaction mixture was stored at −20° C. until further use.

The product was extracted with ethyl acetate (EtOAc) from the cell suspension. The organic layer was dried over MgSO₄ and the solvent was evaporated. The crude extract was subjected to column chromatography (stationary phase: silica 60; solvent:cyclohexane/EtOAc 4:1) to yield the purified 4-acetoxy-2-methylene-butan-1-ol (III). The solvent of fractions containing compound III was evaporated to give pure III, the yield of which is reported in Table 5 herein below. The fractions were analyzed by TLC (cyclohexane:EtOAc, 2:1; detection by KMnO₄ staining). Spectroscopic data was compared to spectroscopic data in literature ([6], [7]).

For the analysis of the whole-cell biotransformation, 250 μL of reaction mixture were acidified with 25 μL of 2 M HCl and extracted with 250 μL EtOAc containing 1 mM of methylbenzoate as internal standard by vigorous shaking. Phase separation was achieved by centrifugation (16,000×g, 4° C., 7 min). The organic phase was dried over MgSO₄. 200 μL of extract were directly subjected to GC-MS or GC-FID analysis for qualitative or quantitative analysis, respectively.

The yields of the reaction reported in Table 5 are represented as the mean of the triplicate reactions together with the standard deviation (SD).

TABLE 5

| | | | | | Standard |
|---|---|---|---|---|---|
| | | | AlkL Co- | Yield | Deviation |
| Identifier | Insert | Gene Origin | expression | (mM)¹ | (mM) |
| pBT10_AlkBmutant | PpGPo1AlkB(I223V) | P. Putida GPo1 | No | 0.81 | 0.05 |
| pBT10_AlkBmutant | PpGPo1AlkB(I223V) | P. Putida GPo1 | Yes | 0.87 | 0.14 |
| pBT10_AlkBmutant | PpGPo1AlkB(F164L) | P. Putida GPo1 | No | 0.87 | 0.05 |

¹Yields reported based on 5 mM of the starting substrate, isoprenyl acetate (II)

### Example 5

### 5a) Cloning and Expression of Homologues of AlkB

The following homologues (a), b), c)) were independently cloned analogously as described by Schrewe et al. [1] and van Nuland et al. [2] into the broad-host vector pCom10_AlkL [3] by excising AlkL and giving the vector pBT10: a) AlkB from Pseudomonas putida P1 (PpP1_AlkB; Accession number: P12691.1) having 90% protein sequence identity with AlkB from P. putida GPo1 (PpGPo1); b) AlkMO from Marinobacter hydrocarbonclasticus (Mh_AlkMO; RCW72391.1) having 78% protein sequence identity with AlkB from P. putida GPo1; and, c) AlkB1 from Alcanivorax borkumensis (Ab_AlkB1; AB110225.1) having 77% protein sequence identity with AlkB from P. putida GPo1.

Synthetic DNA-fragments encoding alkBFG, alkT and, independently, the aforementioned homologues were obtained from Integrated DNA Technologies. The alkBFGT and alkT fragments were sub-cloned into the pJET1.2 vector using the Thermo Scientific CloneJET PCR Cloning Kit. The alkBFG and alkT DNA fragments were inserted into the pCom10 vector backbone via the FastCloning method [4] resulting in the pBT10 plasmid. The homologues a) to c) were inserted into the pBT10 backbone by replacing the alkB gene from P. putida GPO1. Correct assembly of pBT10_AlkBhomolog was verified by colony PCR as well as by Sanger Sequencing.

E. coli BL21(DE3) harboring the pBT10_AlkBhomolog plasmids were independently used to express AlkFGT and said homologues at similar conditions as described in literature ([1], [2]). E. coli BL21(DE3) pCom10_AlkL was used as the empty vector control. Strains were grown in either Luna-Bertani (LB) or M9 Minimal Medium (1×M9 salts, 0.5% glucose, 2 mM MgSO₄, 1 ml L¹ of US^{Fe} trace elements) supplemented with 50 μg mL⁻¹ kanaymycin for selection. 5 mL of LB medium were inoculated with a single colony and incubated overnight at 30° C. and constant agitation (120 rpm). 500 μL of the overnight culture (ONC) were transferred to 50 mL of M9 Minimal Medium in a 100 mL shake flask and the culture was again incubated overnight at 30° C.

For the expression, from 100 to 400 mL of M9 Minimal Medium were inoculated with the M9-preculture to an optical density, as measured at a wavelength of 600 nm (OD₆₀₀), of 0.15 and grown at 30° C. until an OD₆₀₀ of from 0.4 to 0.5 was achieved. By addition of 0.05% (v/v) dicyclopropyl ketone (DCPK), recombinant gene expression was induced. After 4 hours of expression at 30° C., the cells were harvested by centrifugation (4400×g, 4° C., 15 min).

5b) Whole-Cell Biotransformation and Isolation of 4-acetoxy-2-methylene-butan-1-ol (III)

After expression and harvest, each of the three modified cell types were independently re-suspended in the resting cell buffer (50 mM KPi, pH 7.4, 1% glucose, 2 mM MgSO₄) to an OD₆₀₀ of 10. The cells were adapted to the reaction conditions for 5 to 10 minutes. The reaction described below was then performed in biological triplicate.

The reaction was performed in a 1.5 mL glass vial in a total volume of 300 μL. The reaction was started by the addition of 5 mM of the substrate (isoprenyl acetate (II), stock 200 mM in EtOH, 2.5% (v/v) EtOH in the reaction). The cells were independently incubated for 24 hours at 25° C. and under constant agitation (180 rpm). To stop the reaction, the mixture was stored at −20° C. until further use. Substrate conversion was analyzed by GC-MS and GC-FID.

As a negative control, E. coli pCom10_AlkL. To confirm general activity of the AlkFGT and homologue ((a), b) or c)) system, conversion of n-octane to 1-octanol and further overoxidation to octanal and octanoic acid was performed as model reaction.

To verify the formation of 4-acetoxy-2-methylene-butan-1-ol (III) by NMR spectroscopy, the reaction was scaled up to a total volume of 20 mL and was performed in 100 mL shake conducted for 24 hours of reaction time. The reaction mixture was stored at −20° C. until further use.

The product was extracted with ethyl acetate (EtOAc) from the cell suspension. The organic layer was dried over MgSO₄ and the solvent was evaporated. The crude extract was subjected to column chromatography (stationary phase: silica 60; solvent:cyclohexane/EtOAc 4:1) to yield the purified 4-acetoxy-2-methylene-butan-1-ol (III). The solvent of fractions containing compound III was evaporated to give pure III, the yield thereof being reported in Table 6 herein below. The fractions were analyzed by TLC (cyclohexane:EtOAc, 2:1; detection by KMnO₄ staining). Spectroscopic data was compared to spectroscopic data in literature ([6], [7]).

For the analysis of the whole-cell biotransformation, 250 μL of reaction mixture were acidified with 25 μL of 2 M HCl and extracted with 250 μL EtOAc containing 1 mM of methylbenzoate as internal standard by vigorous shaking. Phase separation was achieved by centrifugation (16,000×g, 4° C., 7 min). The organic phase was dried over MgSO₄. 200 μL of extract were directly subjected to GC-MS or GC-FID analysis for qualitative or quantitative analysis, respectively.

The yields of the reaction are reported in Table 6 herein below. The data is represented as the mean of the triplicate reactions together with the standard deviation.

TABLE 6

| | | | | Standard |
|---|---|---|---|---|
| | | AlkL Co- | Yield | Deviation |
| Identifier | Insert | expression | (mM)¹ | (mM) |
| pBT10_PpP1AlkB | PpP1AlkB | No | 0.50 | 0.04 |
| pBT10_MhAlkMO | MhAlkMO | No | 0.73 | 0.02 |
| pBT10_AbAlkB1 | AbAlkB1 | No | 0.35 | 0.02 |

¹Yields reported based on 5 mM of the starting substrate, isoprenyl acetate (II)

### Example 6

6a) Cloning and Expression of Homologues of AlkB with Co-Expression of Transporter Protein AlkL

The following homologues (a), b), c)) were independently cloned analogously as described by Schrewe et al. [1] and van Nuland et al. [2] into the broad-host vector pCom10_AlkL [3] and giving the vector pBTL10: a) AlkB from Pseudomonas putida P1 (PpP1_AlkB; Accession number: P12691.1) having 90% protein sequence identity with AlkB from P. putida GPo1; b) AlkMO from Marinobacter hydrocarbonclasticus (Mh_AlkMO; RCW72391.1) having 78% protein sequence identity with AlkB from P. putida GPo1; and, c) AlkB1 from Alcanivorax borkumensis (Ab_AlkB1; AB110225.1) having 77% protein sequence identity with AlkB from P. putida GPo1 (PpGPo1_AlkB).

Synthetic DNA-fragments encoding alkBFG, alkT and, independently, the aforementioned homologues were obtained from Integrated DNA Technologies. The gene encoding the transporter AlkL was amplified from the pCom10_AlkL plasmid. The alkBFGT and alkT fragments were sub-cloned into the pJET1.2 vector using the Thermo Scientific CloneJET PCR Cloning Kit. The DNA fragments alkBFG, alkL and alkT were inserted into the pCom10 vector backbone via the FastCloning method [4] resulting in the pBTL10 plasmid. The homologues a) to c) were inserted into the pBTL10 backbone by replacing the alkB gene from P. putida GPO1. Correct assembly of pBTL10_AlkBhomolog was verified by colony PCR as well as by Sanger Sequencing.

E. coli BL21(DE3) harboring the pBTL10 plasmids were independently used to express AlkFGTL and said homologues at similar conditions as described in literature ([1], [2]). E. coli BL21(DE3) pCom10_AlkL was used as the empty vector control. Strains were grown in either Luna-Bertani (LB) or M9 Minimal Medium (1×M9 salts, 0.5% glucose, 2 mM MgSO₄, 1 ml L⁻¹ of US^{Fe} trace elements) supplemented with 50 μg mL⁻¹ kanaymycin for selection. 5 mL of LB medium were inoculated with a single colony and incubated overnight at 30° C. and constant agitation (120 rpm). 500 μL of the overnight culture (ONC) were transferred to 50 mL of M9 Minimal Medium in a 100 mL shake flask and the culture was again incubated overnight at 30° C.

For the expression, from 100 to 400 mL of M9 Minimal Medium were inoculated with the M9-preculture to an optical density, as measured at a wavelength of 600 nm (OD₆₀₀), of 0.15 and grown at 30° C. until an OD₆₀₀ of from 0.4 to 0.5 was achieved. By addition of 0.05% (v/v) dicyclopropyl ketone (DCPK), recombinant gene expression was induced. After 4 hours of expression at 30° C., the cells were harvested by centrifugation (4400×g, 4° C., 15 min). 6b) Whole-cell biotransformation and isolation of 4-acetoxy-2-methylene-butan-1-ol

After expression and harvest, each of the three modified cell types were independently re-suspended in the resting cell buffer (50 mM KPi, pH 7.4, 1% glucose, 2 mM MgSO₄) to an OD₆₀₀ of 10. The cells were adapted to the reaction conditions for 5 to 10 minutes. The reaction described below was then performed in biological triplicate.

The reaction was performed in a 1.5 mL glass vial in a total volume of 300 μL. The reaction was started by the addition of 5 mM of the substrate (isoprenyl acetate (II), stock 200 mM in EtOH, 2.5% (v/v) EtOH in the reaction). The cells were independently incubated for 24 hours at 25° C. and under constant agitation (180 rpm). To stop the reaction, the mixture was stored at −20° C. until further use. Substrate conversion was analyzed by GC-MS and GC-FID.

As a negative control, E. coli pCom10_AlkL. To confirm general activity of the AlkFGTL and homologue ((a), b) or c)) system, conversion of n-octane to 1-octanol and further overoxidation to octanal and octanoic acid was performed as model reaction.

To verify the formation of 4-acetoxy-2-methylene-butan-1-ol (III) by NMR spectroscopy, the reaction was scaled up to a total volume of 20 mL and was performed in 100 mL shake conducted for 24 hours of reaction time. The reaction mixture was stored at −20° C. until further use.

The product was extracted with ethyl acetate (EtOAc) from the cell suspension. The organic layer was dried over MgSO₄ and the solvent was evaporated. The crude extract was subjected to column chromatography (stationary phase: silica 60; solvent:cyclohexane/EtOAc 4:1) to yield the purified 4-acetoxy-2-methylene-butan-1-ol (III). The solvent of fractions containing compound III was evaporated to give pure III, the yield of which is reported in Table 7 below. The fractions were analyzed by TLC (cyclohexane:EtOAc, 2:1; detection by KMnO₄ staining). Spectroscopic data was compared to spectroscopic data in literature ([6], [7]).

For the analysis of the whole-cell biotransformation, 250 μL of reaction mixture were acidified with 25 μL of 2 M HCl and extracted with 250 μL EtOAc containing 1 mM of methylbenzoate as internal standard by vigorous shaking. Phase separation was achieved by centrifugation (16,000×g, 4° C., 7 min). The organic phase was dried over MgSO₄. 200 μL of extract were directly subjected to GC-MS or GC-FID analysis for qualitative or quantitative analysis, respectively.

The yields of the reaction are reported in Table 7 herein below. The data is represented as the mean of the triplicate reactions together with the standard deviation (SD).

TABLE 7

| | | | | Standard |
|---|---|---|---|---|
| | | AlkL Co- | Yield | Deviation |
| Identifier | Insert | expression | (mM)¹ | (mM) |
| pBTL10_PpP1AlkB | PpP1AlkB | Yes | 1.07 | 0.06 |
| pBTL10_MhAlkMO | MhAlkMO | Yes | 1.20 | 0.03 |
| pBTL10_AbAlkB1 | AbAlkB1 | Yes | 0.49 | 0.07 |

¹Yields reported based on 5 mM of the starting substrate, isoprenyl acetate (II)

### Example 7: Synthesis of 4-acetoxy-2-methylene butan-1-al

Conversion of the 4-acetoxy-2-methylene-butan-1-ol (III) of the prior step to 4-acetoxy-2-methylene-butanal (IV) was achieved by chemical oxidation using the Jones reagent. The Jones reagent was synthesized from CrO₃ and concentrated H₂SO₄. The crude extract of 4-acetoxy-2-methylene-butan-1-ol (III) (615 mg, containing approximately 0.7 mmol of (III)) was dissolved in 6 mL acetone in a round bottom flask equipped with magnetic stirring and put on ice: Jones reagent (0.345 mmol, 0.5% eq.) was added dropwise thereto. The reaction was followed by TLC. After 45 minutes the reaction was stopped by addition of 2-propanol and the reaction mixture was first washed with double distilled water (ddH₂O) and then extracted with EtOAc. The organic layer was washed twice with a mixture (1:2) of brine and saturated NaHCO₃ and then dried over NaSO₄. The solvent was evaporated and the obtained residue was submitted to NMR spectroscopy to confirm the formation of 4-acetoxy-2-methylene-butanal (IV).

¹H NMR (500 MHz, CDCl₃): δ 9.51 (s, 1H), 6.31 (s, 1H), 6.07 (s, 1H), 4.15 (t, J=6.6 Hz, 2H), 2.56 (t, J=6.5 Hz, 2H), 1.99 (s, 3H) ppm.

### Example 8: Synthesis of 4-acetoxy-2-methylene butyric acid (V)

In a 10 mL round flask equipped with magnetic stirring, 4-hydroxy-2-methylene butyric acid (200 mg, 1.7 mmol) was acetylated with acetic anhydride (5.0 eq.) in pyridine (5.7 eq.) at 0° C., overnight. The reaction was monitored by thin layer chromatography (TLC): once the reaction was completed, the reagents were initially removed by evaporation in vacuo. The obtained residue was washed with 1 M HCl (5 mL) and extracted with EtOAc (3 times, 5 mL). The combined organic layers were concentrated under reduced pressure in a water bath at 50° C. Remaining traces of the reagents were removed by evaporation under inert gas and yielded the title compound (V) in pure form as a yellow, viscous oil (167 mg, 62% yield). The title compound was submitted to NMR spectroscopy, the results of which were in full agreement with reported data in the literature.

¹H NMR (300 MHz, CDCl₃): δ 6.37 (s, 1H), 5.73 (s, 1H), 4.23 (t, J=6.6 Hz, 2H), 2.65 (t, J=6.6 Hz, 2H), 2.03 (s, 3H) ppm.

¹³C NMR (300 MHz, CDCl3): δ 171.3, 171.1, 136.0, 129.5, 62.6, 31.1, 20.9.

### Example 9: Lactonization of 4-Acetoxy-2-methylene butyric acid to α-methylene-γ-butyrolactone (Tulipalin A)

10 mg of 4-acetoxy-2-methylene butyric acid was dissolved in HCl (10.0 eq.) and double-distilled water was added to a total volume of 1 mL. The reaction mixture was incubated at 80° C. and 800 rpm. 20 μL samples were taken at the beginning of the incubation, after 3 hours and after 20 hours and were each analysed by thin layer chromatography (TLC) (EtOAc:DCM, 3:1) and by GC-FID. The samples were therefore extracted with 100 μL EtOAc—containing 1 mM methyl benzoate as the internal standard (ISTD)—and further diluted with solvent to 200 μL. Prior to GC-FID analysis the sample was derivatized by silylation with N,O-bis(trimethylsilyl)trifluoroacetamide (BSTFA, 2 μL) and incubation at 60° C. for 1 hour.

### REFERENCES

[1] Y. M. van Nuland et al., Appl. Environ. Microbiol. 2016, 82, 3801-3807.
[2] M. Schrewe et al., Advanced Synthesis & Catalysis 2011, 353, 3485-3495.
[3] M. K. Julsing et al., Appl. Environ. Microbiol. 2012, 78, 5724-5733.
[4] C. Li et al. BMC Biotechnol. 2011, 11, 92.
[5] N. Iranpoor et al., Journal of Sulfur Chemistry 2007, 28, 581-587.
[6] P. Ferraboschi et al. Tetrahedron: Asymmetry 1994, 5, 691-69
[7] Y. Kim et al., Tetrahedron: Asymmetry 2011, 22, 1658-1661.
[8] Koch et al. Applied Environmental Microbiology 2009, 75, 337-344.

In view of the foregoing description and examples, it will be apparent to those skilled in the art that equivalent modifications or combinations thereof can be made without departing from the scope of the claims.

What is claimed is:

1. A process for the preparation of α-methylene-γ-butyrolactone, said process comprising the steps of:
a) acetylating the C1-hydroxyl group of isoprenol to yield isoprenyl acetate;
b) forming 4-acetoxy-2-methylene-butan-1-ol from said isoprenyl acetate by whole cell biotransformation, said step comprising:
i) contacting a cell (CB) with a culture medium containing said isoprenyl acetate or with a culture medium contiguous with an organic phase containing said isoprenyl acetate under conditions that enable the cell to form 4-acetoxy-2-methylene-butan-1-ol from isoprenyl acetate; and,
ii) optionally isolating the resultant 4-acetoxy-2-methylene-butan-1-ol,
wherein said cell (CB) exhibits activity of at least one alkane monooxygenase enzyme which catalyzes the formation of 4-acetoxy-2-methylene-butan-1-ol from isoprenyl acetate;
c) oxidizing said 4-acetoxy-2-methylene-butan-1-ol to yield 4-acetoxy-2-methylene butyric acid; and,
d) converting said 4-acetoxy-2-methylene butyric acid to α-methylene-γ-butyrolactone by hydroxylysis to γ-hydroxy-α-methylenebutyric acid and subsequent cyclization of said γ-hydroxy-α-methylenebutyric acid.

2. The process according to claim 1, wherein said isoprenol of step a) is obtained via a fermentation stage, said stage comprising:
providing a fermentation medium comprising a fermentable carbohydrate; and,
introducing into said medium an inoculant comprising a culture of one or more microorganisms selected from the group consisting of bacteria, moulds and yeasts, wherein said one or more microorganisms is characterized in that it ferments said carbohydrate to form isoprenol.

3. The process according to claim 1, wherein step a) comprises treating isoprenol at a temperature of from 20 to 150° C. with an acetylation reagent selected from the group consisting of acetic acid, acetic anhydride and acetyl chloride.

4. The process according to claim 1, wherein step a) comprises treating isoprenol with an acetylation reagent in the presence of an enzyme capable of catalyzing the acetylation reaction.

5. The process according to claim 1, wherein said cell (CB) possesses the gene of an alkane monoxygenase, optionally as part of an Alk operon.

6. The process according to claim 1, wherein said cell (CB) is genetically modified to exhibit increased activity of an alkane monooxygenase enzyme encoded by a homologue of the AlkB gene from Pseudomonas putida GP01 or
wherein said cell (CB) is genetically modified to exhibit increased activity of an alkane monooxygenase enzyme encoded by the AlkBGT gene cluster from Pseudomonas putida GP01 or
wherein said cell (CB) is genetically modified to exhibit increased activity of an alkane monooxygenase enzyme encoded by the AlkBGTJH gene cluster from Pseudomonas putida GP01 or
wherein said cell (CB) is genetically modified to exhibit increased activity of an alkane monooxygenase enzyme encoded by the AlkBGTJHL gene cluster from Pseudomonas putida G P01.

7. The process according to claim 1, wherein step c) comprises a two-stage oxidation, wherein:
in a first stage 4-acetoxy-2-methylene-butan-1-ol is oxidized to 4-acetoxy-2-methylene-butan-1-al; and,
in a second stage 4-acetoxy-2-methylene-butan-1-al is oxidized to 4-acetoxy-2-methylene-butyric acid.

8. The process according to claim 7, wherein step c) is performed without intermediate isolation of said 4-acetoxy-2-methylene-butan-1-al by reacting said 4-acetoxy-2-methylene-butan-1-ol with a stochiometric excess of an oxidizing agent selected from: chromic acid (H₂CrO₄); Na₂CrO₄; K₂CrO₄; K₂Cr₂O₇; K₂Cr₂O₇; permanganate; and, Jones reagent.

9. The process according to claim 1, wherein step c) is characterized by an enzymatic oxidation process comprising contacting 4-acetoxy-2-methylene butan-1-ol under aerobic conditions with:
at least one enzyme exhibiting oxidizing activity; and,
optionally, at least one mediating compound which enhances the oxidizing activity of the enzyme.

10. The process according to claim 9, wherein the reaction mixture of step c) comprises from 0.001 to 10 mg of said at least one enzyme per kg of 4-acetoxy-2-methylene butan-1-ol.

11. The process according to claim 9, wherein the reaction mixture of step c) comprises from 0.001 to 10 mg of said least one mediating compound per kg of 4-acetoxy-2-methylene butan-1-ol.

12. The process according to claim 1, wherein step c) is performed by a whole cell biotransformation which comprises:
i) contacting a cell (CC) either with a culture medium containing said 4-acetoxy-2-methylene-butan-1-ol or with a culture medium contiguous with an organic phase containing 4-acetoxy-2-methylene-butan-1-ol under conditions that enable the cell to form 4-acetoxy-2-methylene-butyric acid from 4-acetoxy-2-methylene-butan-1-ol; and,
ii) isolation of the resultant 4-acetoxy-2-methylene-butyric acid,
wherein said cell (CC) exhibits activity of at least one enzyme exhibiting oxidizing activity.

13. The process according to claim 9, wherein said at least one enzyme exhibiting oxidizing activity is selected from the group consisting of: alcohol dehydrogenase (ADH); alcohol oxidase (AlcOx); aldehyde dehydrogenases (AlDH); and, laccase.

14. The process according to claim 1, wherein step c) is performed by a whole cell biotransformation which comprises:
i) contacting a cell (CC1) with a culture medium containing 4-acetoxy-2-methylene-butan-1-ol or with a culture medium contiguous with an organic phase containing 4-acetoxy-2-methylene-butan-1-ol under conditions that enable the cell to form 4-acetoxy-2-methylene-butan-1-al from 4-acetoxy-2-methylene-butan-1-ol;
ii) optionally isolating the resultant 4-acetoxy-2-methylene-butan-1-al;
iii) contacting a cell (CC2) with a culture medium containing said 4-acetoxy-2-methylene-butan-1-al or with a culture medium contiguous with an organic phase containing said 4-acetoxy-2-methylene-butan-1-al under conditions that enable the cell to form 4-acetoxy-2-methylene butyric acid from 4-acetoxy-2-methylene-butan-1-al; and,
iv) isolating the resultant 4-acetoxy-2-methylene butyric acid,
wherein said cells (CC1, CC2) each exhibit increased activity of at least one enzyme exhibiting oxidizing activity.

15. The process according to claim 14, wherein:
said cell (CC1) is genetically modified to exhibit increased activity of at least one of enzyme exhibiting oxidizing activity selected from the group consisting of alcohol dehydrogenase (ADH), alcohol oxidase (AlcOx) and laccase; and,
said cell (CC2) is genetically modified to exhibit increased activity of at least one of enzyme exhibiting oxidizing activity selected from the group consisting of aldehyde dehydrogenase (AlDH) and laccase.

16. The process according to claim 13, wherein:
said alcohol dehydrogenase (ADH) enzyme is encoded by a homologue of the AlkJ gene from Pseudomonas putida GP01; and/or,
said aldehyde dehydrogenase (AlDH) enzyme is encoded by a homologue of the AlkH gene from Pseudomonas putida GP01.

17. The process according to claim 1, wherein step d) comprises subjecting said 4-acetoxy-2-methylene butyric acid to acidic conditions at a temperature of from 30 to 300° C.

18. A process for the preparation of 4-acetoxy-2-methylene-butan-1-ol, said process comprising the steps of:
a) acetylating the C1-hydroxyl group of isoprenol to yield isoprenyl acetate;
b) forming 4-acetoxy-2-methylene-butan-1-ol from said isoprenyl acetate by whole cell biotransformation, said step comprising:
i) contacting a cell (CB) with a culture medium containing said isoprenyl acetate or with a culture medium contiguous with an organic phase containing said isoprenyl acetate under conditions that enable the cell to form 4-acetoxy-2-methylene-butan-1-ol from isoprenyl acetate; and,
ii) isolating the resultant 4-acetoxy-2-methylene-butan-1-ol,
wherein said cell (CB) exhibits activity of at least one alkane monooxygenase enzyme which catalyzes the formation of 4-acetoxy-2-methylene-butan-1-ol from isoprenyl acetate.

19. A process for the preparation of 2-methylenebutan-1,2-diol, said process comprising the steps of:
a) acetylating the C1-hydroxyl group of isoprenol to yield isoprenyl acetate;
b) forming 4-acetoxy-2-methylene-butan-1-ol from said isoprenyl acetate by whole cell biotransformation, said step comprising:
i) contacting a cell (CB) with a culture medium containing said isoprenyl acetate or with a culture medium contiguous with an organic phase containing said isoprenyl acetate under conditions that enable the cell to form 4-acetoxy-2-methylene-butan-1-ol from isoprenyl acetate; and,
ii) isolating the resultant 4-acetoxy-2-methylene-butan-1-ol,
wherein said cell (CB) exhibits activity of at least one alkane monooxygenase enzyme which catalyzes the formation of 4-acetoxy-2-methylene-butan-1-ol from isoprenyl acetate; and,
c) hydrolysis of said 4-acetoxy-2-methylene-butan-1-ol to form 2-methylenebutan-1,2-diol.

20. A process for preparing a compound of Formula (BIII) from a compound of Formula (BII) by whole cell biotransformation: 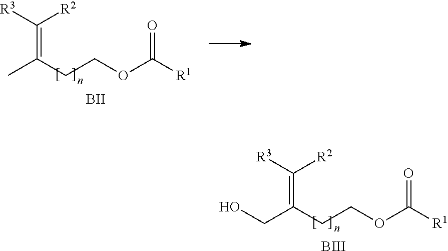
wherein:
n is an integer of from 0 to 8;
R¹ is C₁-C₄ alkyl;
R² is H or C₁-C₄ alkyl; and,
R³ is H or C₁-C₄ alkyl,
said process comprising:
i) contacting a cell (CB) with a culture medium containing said compound of Formula (BII) or with a culture medium contiguous with an organic phase containing said compound of Formula (BII) under conditions that enable the cell to form said compound of Formula (BIII) from said compound of Formula (BII); and,
ii) isolating the resultant compound of Formula (BIII),
wherein said cell (CB) exhibits activity of at least one alkane monooxygenase enzyme which catalyzes the Cₙ₊₃-hydroxylation of said compound of Formula (BII).

21. The process according to claim 18, wherein said cell (CB) possesses the gene of an alkane monoxygenase, optionally as part of an Alk operon.

22. The process according to claim 18, wherein said cell (CB) is genetically modified to exhibit increased activity of an alkane monooxygenase enzyme encoded by a homologue of the AlkB gene from Pseudomonas putida GP01 or
wherein said cell (CB) is genetically modified to exhibit increased activity of an alkane monooxygenase enzyme encoded by the AlkBGT gene cluster from Pseudomonas putida GP01 or
wherein said cell (CB) is genetically modified to exhibit increased activity of an alkane monooxygenase enzyme encoded by the AlkBGTJH gene cluster from Pseudomonas putida GP01 or
wherein said cell (CB) is genetically modified to exhibit increased activity of an alkane monooxygenase enzyme encoded by the AlkBGTJHL gene cluster from Pseudomonas putida G P01.

23.
